(19) United States
(12) Patent Application Publication
(10) Pub. No.: US 2019/0382709 A1
(43) Pub. Date: Dec. 19, 2019

(54) Automated Production and Collection

(71) Applicant: Terumo BCT, Inc., Lakewood, CO (US)

(72) Inventors: Boah VANG, Northglenn, CO (US); Brian J. NANKERVIS, Golden, CO (US)

(73) Assignee: Terumo BCT, Inc., Lakewood, CO (US)

(21) Appl. No.: 16/097,763

(22) PCT Filed: May 5, 2017
(86) PCT No.: PCT/US2017/031409

§ 371 Date: Oct. 30, 2018

Related U.S. Application Data
(60) Provisional application No. 62/332,426, filed on May 5, 2016.
(60) Provisional application No. 62/333,013, filed on May 6, 2016.
(60) Provisional application No. 62/500,962, filed on May 3, 2017.

(51) Int. Cl. C12M 1/36; C12P 21/02; C12M 1/12; C12M 1/00; C12M 1/26

(52) U.S. Cl. CPC C12M 41/48; C12P 21/02; C12M 25/02; C12M 23/14; C12M 37/06; C12M 25/10; C12M 29/16; C12M 29/04; C12M 33/00

(57) ABSTRACT

Embodiments described herein provide for the production, isolation, and/or collection of cellular product(s) released or secreted from cells. Cells may be expanded in the intracapillary (or extracapillary) space of a bioreactor of a cell expansion system with media. Cells may release cellular products into the fluid space of the bioreactor. Examples of such released cellular products include extracellular particles, such as extracellular vesicles (EVs). To collect the extracellular particles released from the cells being expanded, as opposed to any extracellular particles from other sources, a washout procedure may be used to eliminate any serum proteins prior to collecting the released extracellular particles from the expanding cells. The released cellular products may be collected or concentrated through the control of outlet parameters, while nutrients may reach the cells through the diffusion of media through a semi-permeable membrane, for example. The released cellular products may then be harvested.

### CROSS-REFERENCE TO RELATED APPLICATIONS

This application claims priority to, and the benefit of, U.S. Provisional Application Ser. No. 62/332,426, filed on May 5, 2016, and entitled “Automated Production and Collection;” U.S. Provisional Application Ser. No. 62/333,013, filed on May 6, 2016, and entitled “Automated Production and Collection;” and U.S. Provisional Application Ser. No. 62/500,962, filed on May 3, 2017, and entitled “Automated Production and Collection.” The disclosures of the above-identified applications are hereby incorporated herein by reference in their entireties as if set forth herein in full for all that they teach and for all purposes.

### BACKGROUND

Cell Expansion Systems (CESs) are used to expand and differentiate cells. Cell expansion systems may be used to expand, e.g., grow, a variety of adherent and suspension cells. For example, cell expansion systems may be used to expand mesenchymal stem cells (MSCs) and other types of cells, such as bone marrow cells. Stem cells which are expanded from donor cells may be used to repair or replace damaged or defective tissues and have broad clinical applications for a wide range of diseases. Cells, of both adherent and non-adherent type, may be grown in a bioreactor in a cell expansion system.

### SUMMARY

Embodiments of the present disclosure generally relate to producing, isolating, and/or collecting cellular product(s) released or secreted from cells. Such released or secreted cellular products may be referred to as released or secreted agent(s), released or secreted constituent(s), cellular produced agent(s), cellular produced constituent(s), released or secreted particle(s), released or secreted molecule(s), extracellular particle(s), released or secreted protein(s), transfer mechanism(s), etc. Examples of such extracellular particles include, but are not limited to, extracellular vesicles (EVs), viral vectors, etc.

Extracellular vesicles (EVs) may be produced by cells, and, during cell culture, EVs may be released into the fluid or media within which they are cultured or expanded (often called conditioned media due to the presence of important by-products created during expansion). EVs include exosomes and microvesicles, for example. EVs contain RNA, DNA, and proteins that are essential for cell communication and other important cellular processes. EVs may be isolated from body fluids such as serum, plasma, urine, and cell culture supernatant, for example.

In embodiments, intercellular communication plays an important function in cell biology. A cell's ability to communicate with other cells enables complex mechanisms such as protein synthesis to occur. There are a number of ways that cells can communicate with each other such as direct cell-cell contact or transfer of secreted molecules, for example. EVs, such as microvesicles and exosomes, have the ability to mediate intracellular communication and facilitate the transfer of genetic information. Microvesicles are direct buds from plasma membranes and often contain surface markers similar to the membrane of origin. Exosomes may be formed when vesicular endosomes fuse with plasma membranes and bud off into the extracellular space. Due to their active role in genetic information transfer, microvesicles and exosomes can be used in therapeutic applications. For example, EVs may act as antigen-presenting cells to stimulate immune responses, and microvesicles may transfer and activate chemokine receptors resulting in anti-apoptotic effects.

In embodiments, a cell expansion system may be used to expand cells. Such expansion may occur through the use of a bioreactor or cell growth chamber. In an embodiment, such bioreactor or cell growth chamber comprises a hollow fiber membrane, for example. Such hollow fiber membrane may include an extracapillary (EC) space and an intracapillary (IC) space. A cell expansion system may expand a variety of cell types, such as mesenchymal stem cells, cancer cells, T-cells, fibroblasts, and myoblasts. Each of these cell types may release EVs into the fluid space of a bioreactor which may then be collected via an outlet bag. The semi-permeable hollow fibers of a bioreactor allow essential nutrients (e.g., glucose) to reach the cells and metabolic waste products (e.g., lactate) to exit the system via diffusion. Cells may be retained on the intracapillary side of the hollow fibers while EVs may be allowed to concentrate in the fluid space and may then be harvested from the system without harvesting the cells, unless it is desired to also harvest the cells.

Embodiments of the present disclosure further relate to using an automated washout procedure to remove serum proteins used to culture or expand the cells prior to the collection of the released cellular product(s), e.g., EVs or viral vectors, etc., from the expanding cells. Such washout procedure allows for the system to purify the released cellular product(s) by first removing any released cellular product(s), e.g., EVs or viral vectors, etc., from any serum or other source(s) used to expand the cells before beginning the collection of released cellular product(s) from the cells being expanded.

Embodiments of the present disclosure further provide for enabling the collection or concentrating of released cellular product(s) through the use of the multi-compartment bioreactor. By controlling the outlet parameters, such as by closing an IC outlet valve (keeping the EC outlet open), for example, released cellular product(s) may increase in concentration on the IC side while nutrients, e.g., glucose, are still able to reach the cells on the IC side through the addition of media on the EC side and diffusion through the membrane. Such collection may continue for a period of time, such as for about twenty-four (24) hours to about seventy-two (72) hours, for example. In an embodiment, such collection continues for about forty-eight (48) hours. After allowing such concentration of released cellular product(s) to increase, the released cellular product(s) may be harvested into a harvest bag or other container. Attached cells may remain in the bioreactor during such harvest process until it may be desired to release and harvest such cells, if at all, according to an embodiment.

Embodiments of the present disclosure provide for implementing such production and/or collection of released cellular product(s) through the use of one or more protocols or tasks for use with a cell expansion system. Such protocols or tasks may include pre-programmed protocols or tasks. In other embodiments, such protocols or tasks may include custom or user-defined protocols or tasks. Through a user interface (UI) and graphical user interface (GUI) elements, a custom or user-defined protocol or task may be created. A task may comprise one or more steps. In other embodiments, a pre-programmed, default, or otherwise previously saved task may be selected. In yet other embodiments, such production and/or collection may be implemented through the use of one or more manual protocols or tasks for use with a cell expansion system.

This Summary is included to provide a selection of concepts in a simplified form, in which such concepts are further described below in the Detailed Description. This Summary is not intended to be used in any way to limit the claimed subject matter's scope. Features, including equivalents and variations thereof, may be included in addition to those provided herein.

### BRIEF DESCRIPTION OF THE DRAWINGS

Embodiments of the present disclosure may be described by referencing the accompanying figures. In the figures, like numerals refer to like items.

FIG. 1A depicts an embodiment of a cell expansion system (CES).

FIG. 1B illustrates a front elevation view of an embodiment of a bioreactor showing circulation paths through the bioreactor.

FIG. 1C depicts a rocking device for moving a cell growth chamber rotationally or laterally during operation of a cell expansion system, according to embodiments of the present disclosure.

FIG. 2 illustrates a perspective view of a cell expansion system with a pre-mounted fluid conveyance device, in accordance with embodiments of the present disclosure.

FIG. 3 depicts a perspective view of a housing of a cell expansion system, in accordance with embodiments of the present disclosure.

FIG. 4 illustrates a perspective view of a pre-mounted fluid conveyance device, in accordance with embodiments of the present disclosure

FIG. 5 depicts a schematic of a cell expansion system, in accordance with an embodiment of the present disclosure.

FIG. 6 illustrates a schematic of a cell expansion system, in accordance with another embodiment of the present disclosure.

FIG. 7 depicts a flow diagram illustrating the operational characteristics of a process for producing and/or collecting released constituents, in accordance with embodiments of the present disclosure.

FIG. 8 illustrates a flow diagram depicting the operational characteristics of a process for producing and/or collecting released cellular products, in accordance with embodiments of the present disclosure.

FIG. 9 depicts a flow diagram illustrating the operational characteristics of a process for producing and/or collecting released agents, in accordance with embodiments of the present disclosure.

FIG. 10 illustrates an example processing system of a cell expansion system upon which embodiments of the present disclosure may be implemented.

FIG. 11 illustrates an example result of extracting protein from a media in a cell expansion system, in accordance with embodiments of the present disclosure.

FIG. 12 illustrates an example result of using a cell expansion system to generate EVs, in accordance with embodiments of the present disclosure.

FIG. 13A illustrates an example result of using a cell expansion system to generate EVs, in accordance with embodiments of the present disclosure.

FIG. 13B illustrates an example result of using a cell expansion system to generate EVs, in accordance with embodiments of the present disclosure.

FIG. 13C illustrates an example result of using a cell expansion system to generate EVs, in accordance with embodiments of the present disclosure.

FIG. 14 illustrates an example result of using a cell expansion system to generate EVs, in accordance with embodiments of the present disclosure.

### DETAILED DESCRIPTION

The following Detailed Description provides a discussion of illustrative embodiments with reference to the accompanying drawings. The inclusion of specific embodiments herein should not be construed as limiting or restricting the present disclosure. Further, while language specific to features, acts, and/or structures, for example, may be used in describing embodiments herein, the claims are not limited to the features, acts, and/or structures described. A person of skill in the art will appreciate that other embodiments, including improvements, are within the spirit and scope of the present disclosure. Further, any alternatives or additions, including any listed as separate embodiments, may be used or incorporated with any other embodiments herein described.

Embodiments of the present disclosure are generally directed to systems and methods for producing, isolating, and/or collecting released cellular product(s), e.g., extracellular vesicles (EVs), viral vectors, etc., in a cell expansion system. Embodiments of the present disclosure further provide for enabling the collection or concentrating of released cellular product(s) through the use of a multi-compartment bioreactor, for example.

In embodiments, the permeability of a hollow fiber membrane allows the separation of cells from other constituents by retaining cells in the intracapillary (IC) loop, for example, while soluble molecules may pass freely into the extracapillary (EC) loop, for example, thereby eliminating an additional isolation step. Metabolic demands of cells in culture (e.g., glucose, lactate, amino acids, vitamins) may be met using media added to the EC side of the bioreactor. Such media may be diffused through the semi-permeable membrane(s) of the bioreactor. Media constituents with molecular weights too large to diffuse through the membrane(s) may be added to the IC side of the bioreactor using ultrafiltration (either continuously or intermittent bolus additions, for example). In an embodiment, ultrafiltration (IC outlet valve closed) may be used in order to maintain any constituents too large to diffuse through the membrane on the IC side of the bioreactor. EVs produced by the cells may not be able to diffuse through the membrane, i.e., their molecular weights may be too large. EVs may therefore be maintained on the IC side of the bioreactor during expansion (or defined collection period) where the EV concentration may be continuously increased. The EVs may then be harvested from the IC side of the bioreactor to a harvest container(s) or harvest bag(s) at defined intervals or at the end of the entire process, for example. Without the benefit of the two fluid compartments of the hollow fiber membrane, the EV concentration or collection may be limited to the rate at which cells produce EVs and the rate fresh media may be added to the culture environment to satisfy nutrient demands, according to embodiments.

By controlling outlet parameters, such as by closing an IC outlet valve (keeping the EC outlet open), for example, released cellular product(s) may therefore increase in concentration on the IC side while nutrients, e.g., glucose, are still able to reach the cells on the IC side through the addition of media on the EC side and diffusion through the membrane. In other embodiments, nutrients may feed the cells on the IC side through the addition of media on the IC side. In further embodiments, cell expansion may occur on the EC side with an addition of media on the IC side (or EC side) and diffusion through the membrane to reach the cells. The collection of EVs may continue for a period of time, such as for about twenty-four (24) hours or more. In other embodiments, such collection may continue for less than about twenty-four (24) hours. In other embodiments, such collection may continue for about forty-eight (48) hours to about seventy-two (72) hours, for example. After allowing such concentration of released cellular product(s) to increase, the released cellular product(s) may be harvested into a harvest bag or other container. Attached cells may remain in the bioreactor during such harvest process until it is desired to release and harvest such cells, if at all, according to an embodiment.

Embodiments are directed to a cell expansion system, as noted above. In embodiments, such cell expansion system is closed, in which a closed cell expansion system comprises contents that are not directly exposed to the atmosphere. Such cell expansion system may be automated. In embodiments, cells, of both adherent and non-adherent or suspension type, may be grown in a bioreactor in the cell expansion system. According to embodiments, the cell expansion system may include base media or other type of media. Methods for replenishment of media are provided for cell growth occurring in a bioreactor of the closed cell expansion system. In embodiments, the bioreactor used with such systems may be a hollow fiber bioreactor. Many types of bioreactors may be used in accordance with embodiments of the present disclosure.

The system may include, in embodiments, a bioreactor that further includes a first fluid flow path having at least opposing ends, a first opposing end of the first fluid flow path fluidly associated with a first port of a hollow fiber membrane and a second end of the first fluid flow path fluidly associated with a second port of the hollow fiber membrane, in which the first fluid flow path comprises an intracapillary portion of the hollow fiber membrane. In embodiments, a hollow fiber membrane comprises a plurality of hollow fibers. The system may further include a fluid inlet path fluidly associated with the first fluid flow path, in which a plurality of cells is introduced into the first fluid flow path through a first fluid inlet path. A first pump for circulating fluid in the first fluid flow path of the bioreactor may also be included. In embodiments, the system includes a controller for controlling operation of the first pump. In an embodiment, the controller is a computing system, including a processor, for example. The controller is configured, in embodiments, to control the pump to circulate a fluid at a first rate within the first fluid flow path. In some embodiments, a second pump for transferring intracapillary inlet fluid from an intracapillary media bag to the first fluid flow path and a second controller for controlling operation of the second pump are included. The second controller, in embodiments, controls the second pump to transfer cells from a cell inlet bag to the first fluid flow path, for example. Additional controllers, e.g., third controller, fourth controller, fifth controller, sixth controller, etc., may be used in accordance with embodiments. Further, additional pumps, e.g., third pump, fourth pump, fifth pump, sixth pump, etc., may be used in accordance with embodiments of the present disclosure. In addition, while the present disclosure may refer to a media bag, a cell inlet bag, etc., multiple bags, e.g., a first media bag, a second media bag, a third media bag, a first cell inlet bag, a second cell inlet bag, a third cell inlet bag, etc., and/or other types of containers, may be used in embodiments. In other embodiments, a single media bag, a single cell inlet bag, etc., may be used. Further, additional or other fluid paths, e.g., a second fluid flow path, a second fluid inlet path, etc., may be included in embodiments.

In other embodiments, the system is controlled by, for example: a processor coupled to the cell expansion system; a display device, in communication with the processor, and operable to display data; and a memory, in communication with and readable by the processor, and containing a series of instructions. In embodiments, when the instructions are executed by the processor, the processor receives an instruction to coat the bioreactor, for example. In response to the instruction to coat the bioreactor, the processor may execute a series of steps to coat the bioreactor and may next receive an instruction to load cells into the bioreactor, for example. In response to the instruction to load cells, the processor may execute a series of steps to load the cells from a cell inlet bag, for example, into the bioreactor.

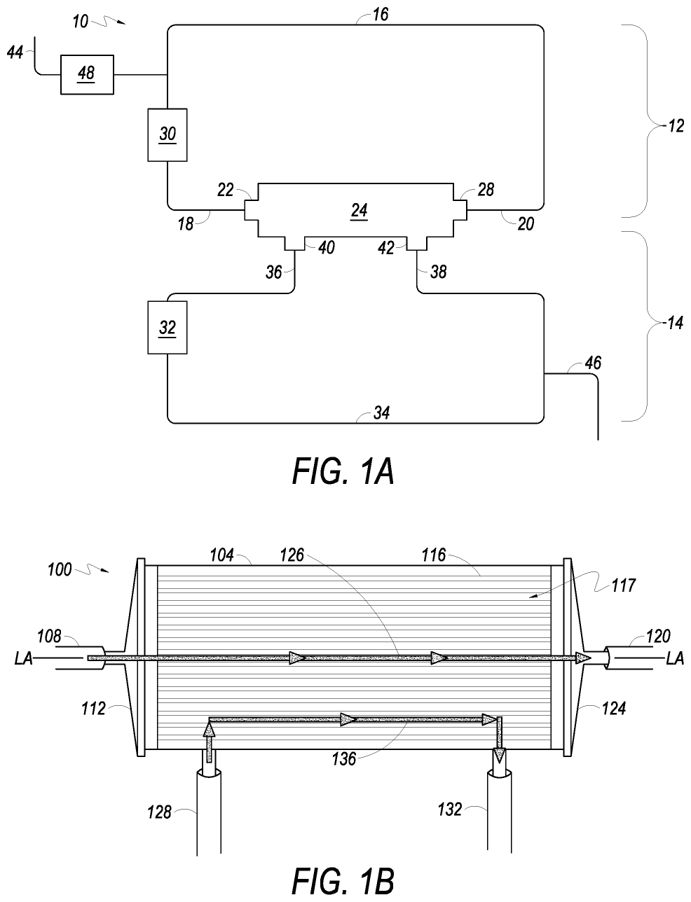

A schematic of an example cell expansion system (CES) is depicted in Figure (FIG. 1A, in accordance with embodiments of the present disclosure. “CES” and “system” may be used interchangeably. CES 10 includes first fluid circulation path 12 and second fluid circulation path 14. First fluid flow path 16 has at least opposing ends 18 and 20 fluidly associated with a hollow fiber cell growth chamber 24 (also referred to herein as a “bioreactor”), according to embodiments. Specifically, opposing end 18 may be fluidly associated with a first inlet 22 of cell growth chamber 24, and opposing end 20 may be fluidly associated with first outlet 28 of cell growth chamber 24. Fluid in first circulation path 12 flows through the interior of hollow fibers 116 (see FIG. 1B) of hollow fiber membrane 117 (see FIG. 1B) disposed in cell growth chamber 24 (cell growth chambers and hollow fiber membranes are described in more detail infra). Further, first fluid flow control device 30 may be operably connected to first fluid flow path 16 and may control the flow of fluid in first circulation path 12.

Second fluid circulation path 14 includes second fluid flow path 34, cell growth chamber 24, and a second fluid flow control device 32. The second fluid flow path 34 has at least opposing ends 36 and 38, according to embodiments. Opposing ends 36 and 38 of second fluid flow path 34 may be fluidly associated with inlet port 40 and outlet port 42 respectively of cell growth chamber 24. Fluid flowing through cell growth chamber 24 may be in contact with the outside of hollow fiber membrane 117 (see FIG. 1B) in the cell growth chamber 24, in which a hollow fiber membrane comprises a plurality of hollow fibers. Second fluid circulation path 14 may be operably connected to second fluid flow control device 32.

First and second fluid circulation paths 12 and 14 may thus be separated in cell growth chamber 24 by a hollow fiber membrane 117 (see FIG. 1B). Fluid in first fluid circulation path 12 flows through the intracapillary (“IC”) space of the hollow fibers in the cell growth chamber 24. First circulation path 12 may be referred to as the “IC loop.” Fluid in second circulation path 14 flows through the extracapillary (“EC”) space in the cell growth chamber 24. Second fluid circulation path 14 may be referred to as the “EC loop.” Fluid in first fluid circulation path 12 may flow in either a co-current or counter-current direction with respect to flow of fluid in second fluid circulation path 14, according to embodiments.

Fluid inlet path 44 may be fluidly associated with first fluid circulation path 12. Fluid inlet path 44 allows fluid into first fluid circulation path 12, while fluid outlet path 46 allows fluid to leave CES 10. Third fluid flow control device 48 may be operably associated with fluid inlet path 44. Alternatively, third fluid flow control device 48 may alternatively be associated with first outlet path 46.

Fluid flow control devices as used herein may comprise a pump, valve, clamp, or combination thereof, according to embodiments. Multiple pumps, valves, and clamps can be arranged in any combination. In various embodiments, the fluid flow control device is or includes a peristaltic pump. In embodiments, fluid circulation paths, inlet ports, and outlet ports may be constructed of tubing of any material.

Various components are referred to herein as “operably associated.” As used herein, “operably associated” refers to components that are linked together in operable fashion and encompasses embodiments in which components are linked directly, as well as embodiments in which additional components are placed between the two linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid, but contact other components to manipulate the system (e.g., a peristaltic pump that pumps fluids through flexible tubing by compressing the exterior of the tube).

Generally, any kind of fluid, including buffers, protein containing fluid, and cell-containing fluid, for example, can flow through the various circulations paths, inlet paths, and outlet paths. As used herein, “fluid,” “media,” and “fluid media” are used interchangeably.

Turning to FIG. 1B, an example of a hollow fiber cell growth chamber 100 which may be used with the present disclosure is shown in front side elevation view. Cell growth chamber 100 has a longitudinal axis LA-LA and includes cell growth chamber housing 104. In at least one embodiment, cell growth chamber housing 104 includes four openings or ports: IC inlet port 108, IC outlet port 120, EC inlet port 128, and EC outlet port 132.

According to embodiments of the present disclosure, fluid in a first circulation path enters cell growth chamber 100 through IC inlet port 108 at a first longitudinal end 112 of the cell growth chamber 100, passes into and through the intracapillary side (referred to in various embodiments as the intracapillary (“IC”) side or “IC space” of a hollow fiber membrane) of a plurality of hollow fibers 116 comprising hollow fiber membrane 117, and out of cell growth chamber 100 through IC outlet port 120 located at a second longitudinal end 124 of the cell growth chamber 100. The fluid path between the IC inlet port 108 and the IC outlet port 120 defines the IC portion 126 of the cell growth chamber 100. Fluid in a second circulation path flows in the cell growth chamber 100 through EC inlet port 128, comes in contact with the extracapillary side or outside (referred to as the “EC side” or “EC space” of the membrane) of the hollow fibers 116, and exits cell growth chamber 100 via EC outlet port 132. The fluid path between the EC inlet port 128 and the EC outlet port 132 comprises the EC portion 136 of the cell growth chamber 100. Fluid entering cell growth chamber 100 via the EC inlet port 128 may be in contact with the outside of the hollow fibers 116. Small molecules (e.g., ions, water, oxygen, lactate, etc.) may diffuse through the hollow fibers 116 from the interior or IC space of the hollow fiber to the exterior or EC space, or from the EC space to the IC space. Large molecular weight molecules, such as growth factors, are typically too large to pass through the hollow fiber membrane, and may remain in the IC space of the hollow fibers 116. The media may be replaced as needed, in embodiments. Media may also be circulated through an oxygenator or gas transfer module to exchange gasses as needed. Cells may be contained within a first circulation path and/or a second circulation path, as described below, and may be on either the IC side and/or EC side of the membrane, according to embodiments.

The material used to make the hollow fiber membrane 117 may be any biocompatible polymeric material which is capable of being made into hollow fibers. One material which may be used is a synthetic polysulfone-based material, according to an embodiment of the present disclosure. In order for the cells to adhere to the surface of the hollow fibers, the surface may be modified in some way, either by coating at least the cell growth surface with a protein such as fibronectin (FN) or collagen, or by exposing the surface to radiation, according to embodiments. Gamma treating the membrane surface allows for attachment of adherent cells without additionally coating the membrane with fibronectin, cryoprecipitate, or the like. Bioreactors made of gamma treated membranes may be reused. Other coatings and/or treatments for cell attachment may be used in accordance with embodiments of the present disclosure.

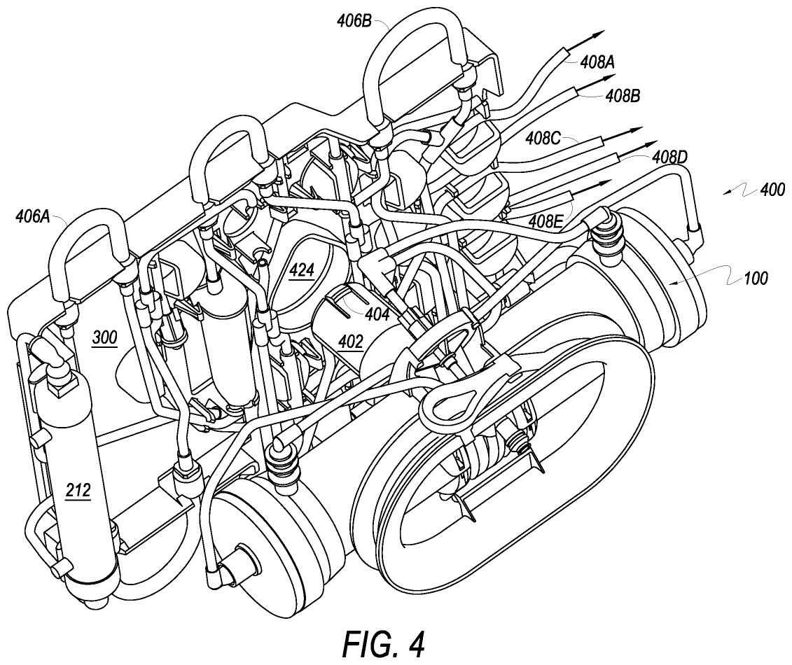

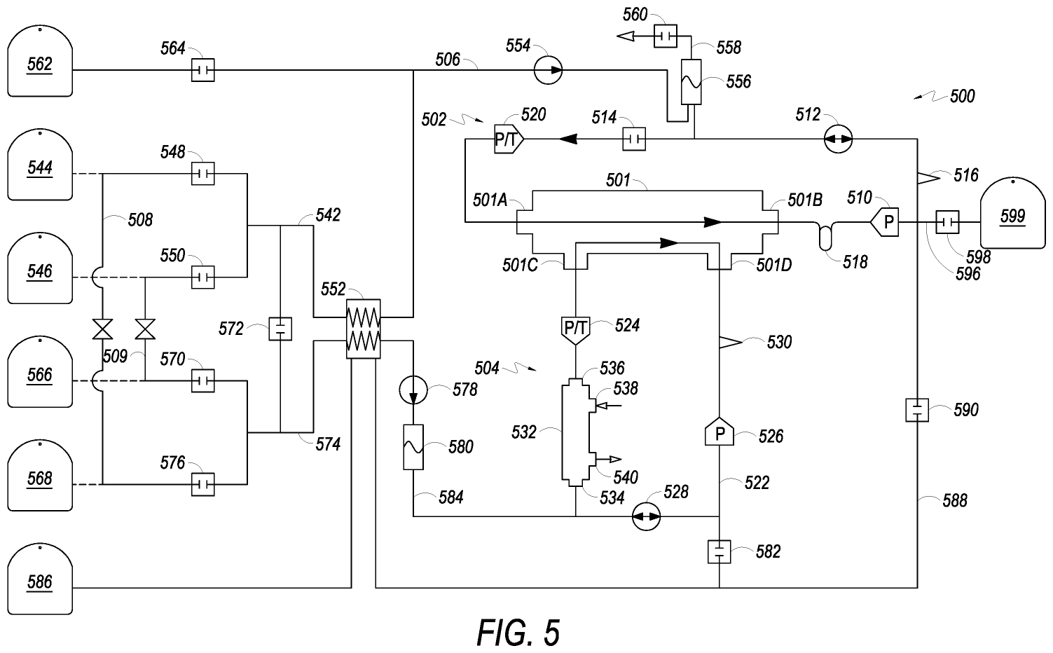

In embodiments, the CES (such as CES 500 (see FIG. 5) and/or CES 600 (see FIG. 6), for example) may include a device configured to move or “rock” the cell growth chamber relative to other components of the cell expansion system by attaching it to a rotational and/or lateral rocking device. FIG. 1C shows one such device, in which a bioreactor 100 may be rotationally connected to two rotational rocking components and to a lateral rocking component, according to an embodiment.

A first rotational rocking component 138 rotates the bioreactor 100 around central axis 142 of the bioreactor 100. Rotational rocking component 138 may be rotationally associated with bioreactor 100. In embodiments, bioreactor 100 may be rotated continuously in a single direction around central axis 142 in a clockwise or counterclockwise direction. Alternatively, bioreactor 100 may rotate in alternating fashion, first clockwise, then counterclockwise, for example, around central axis 142, according to embodiments.

The CES may also include a second rotational rocking component that rotates bioreactor 100 around rotational axis 144. Rotational axis 144 may pass through the center point of bioreactor 100 and may be normal to central axis 142. Bioreactor 100 may be rotated continuously in a single direction around rotational axis 144 in a clockwise or counterclockwise direction, in embodiments. Alternatively, bioreactor 100 may be rotated around rotational axis 144 in an alternating fashion, first clockwise, then counterclockwise, for example. In various embodiments, bioreactor 100 may also be rotated around rotational axis 144 and positioned in a horizontal or vertical orientation relative to gravity.

In embodiments, lateral rocking component 140 may be laterally associated with bioreactor 100. The plane of lateral rocking component 140 moves laterally in the -x and -y directions, in embodiments. The settling of cells in the bioreactor may be reduced by movement of cell-containing media within the hollow fibers, according to embodiments.

The rotational and/or lateral movement of a rocking device may reduce the settling of cells within the device and reduce the likelihood of cells becoming trapped within a portion of the bioreactor. The rate of cells settling in the cell growth chamber is proportional to the density difference between the cells and the suspension media, according to Stoke's Law. In certain embodiments, a 180-degree rotation (fast) with a pause (having a total combined time of 30 seconds, for example) repeated as described above keeps non-adherent red blood cells suspended. A minimum rotation of about 180 degrees would be preferred in an embodiment; however, one could use rotation of up to 360 degrees or greater. Different rocking components may be used separately, or may be combined in any combination. For example, a rocking component that rotates bioreactor 100 around central axis 142 may be combined with the rocking component that rotates bioreactor 100 around axis 144. Likewise, clockwise and counterclockwise rotation around different axes may be performed independently in any combination.

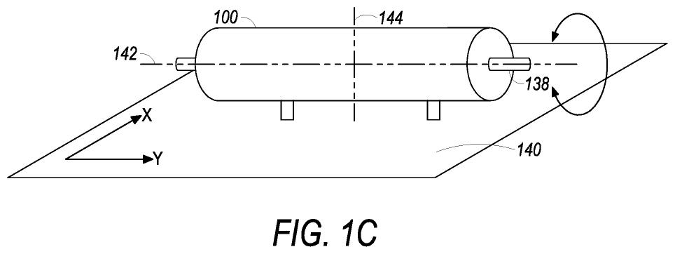

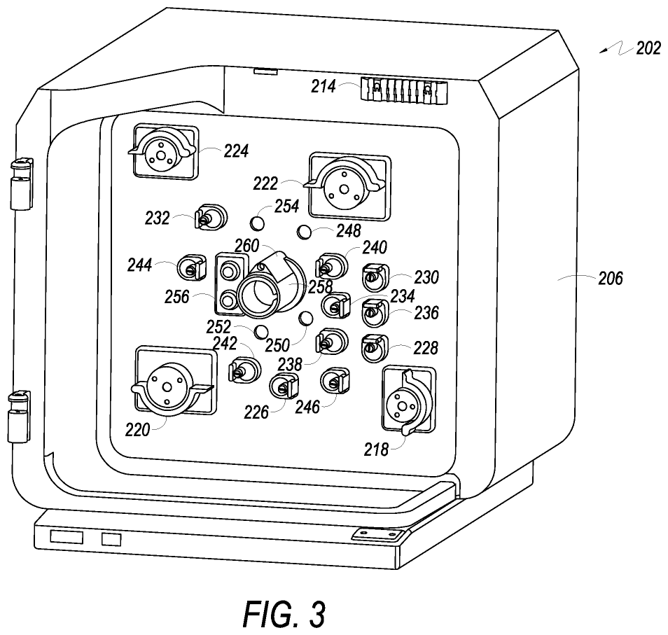

Turning to FIG. 2, an embodiment of a cell expansion system 200 with a pre-mounted fluid conveyance assembly is shown in accordance with embodiments of the present disclosure. The CES 200 includes a cell expansion machine 202 that comprises a hatch or closable door 204 for engagement with a back portion 206 of the cell expansion machine 202. An interior space 208 within the cell expansion machine 202 includes features adapted for receiving and engaging a pre-mounted fluid conveyance assembly 210. The pre-mounted fluid conveyance assembly 210 is detachably-attachable to the cell expansion machine 202 to facilitate relatively quick exchange of a new or unused pre-mounted fluid conveyance assembly 210 at a cell expansion machine 202 for a used pre-mounted fluid conveyance assembly 210 at the same cell expansion machine 202. A single cell expansion machine 202 may be operated to grow or expand a first set of cells using a first pre-mounted fluid conveyance assembly 210 and, thereafter, may be used to grow or expand a second set of cells using a second pre-mounted fluid conveyance assembly 210 without needing to be sanitized between interchanging the first pre-mounted fluid conveyance assembly 210 for the second pre-mounted fluid conveyance assembly 210. The pre-mounted fluid conveyance assembly 210 includes a bioreactor 100 and an oxygenator or gas transfer module 212 (also see FIG. 4). Tubing guide slots are shown as 214 for receiving various media tubing connected to pre-mounted fluid conveyance assembly 210, according to embodiments.

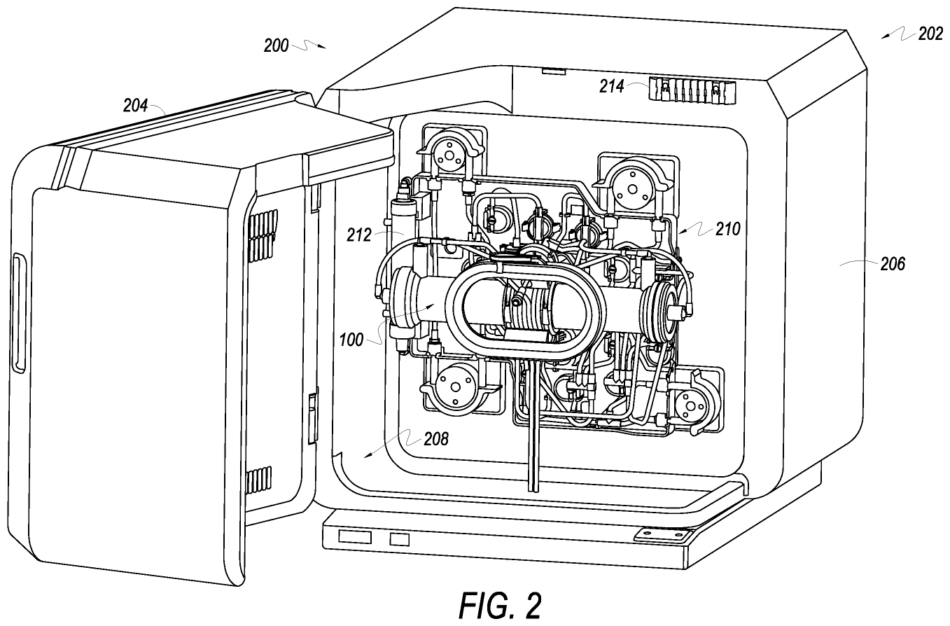

Next, FIG. 3 illustrates the back portion 206 of cell expansion machine 202 prior to detachably-attaching a pre-mounted fluid conveyance assembly 210 (FIG. 2), in accordance with embodiments of the present disclosure. The closable door 204 (shown in FIG. 2) is omitted from FIG. 3. The back portion 206 of the cell expansion machine 202 includes a number of different structures for working in combination with elements of a pre-mounted fluid conveyance assembly 210. More particularly, the back portion 206 of the cell expansion machine 202 includes a plurality of peristaltic pumps for cooperating with pump loops on the pre-mounted fluid conveyance assembly 210, including the IC circulation pump 218, the EC circulation pump 220, the IC inlet pump 222, and the EC inlet pump 224. In addition, the back portion 206 of the cell expansion machine 202 includes a plurality of valves, including the IC circulation valve 226, the reagent valve 228, the IC media valve 230, the air removal valve 232, the cell inlet valve 234, the wash valve 236, the distribution valve 238, the EC media valve 240, the IC waste valve 242, the EC waste valve 244, and the harvest valve 246. Several sensors are also associated with the back portion 206 of the cell expansion machine 202, including the IC outlet pressure sensor 248, the combination IC inlet pressure and temperature sensors 250, the combination EC inlet pressure and temperature sensors 252, and the EC outlet pressure sensor 254. Also shown is an optical sensor 256 for an air removal chamber (ARC), according to an embodiment.

In accordance with embodiments, a shaft or rocker control 258 for rotating the bioreactor 100 is shown. Shaft fitting 260 associated with the shaft or rocker control 258 allows for proper alignment of a shaft access aperture, see e.g., 424 (FIG. 4) of a tubing-organizer, see e.g., 300 (FIG. 4) of a pre-mounted conveyance assembly 210 or 400 with the back portion 206 of the cell expansion machine 202. Rotation of shaft or rocker control 258 imparts rotational movement to shaft fitting 260 and bioreactor 100. Thus, when an operator or user of the CES 200 attaches a new or unused pre-mounted fluid conveyance assembly 400 (FIG. 4) to the cell expansion machine 202, the alignment is a relatively simple matter of properly orienting the shaft access aperture 424 (FIG. 4) of the pre-mounted fluid conveyance assembly 210 or 400 with the shaft fitting 260.

Turning to FIG. 4, a perspective view of a detachably-attachable pre-mounted fluid conveyance assembly 400 is shown. The pre-mounted fluid conveyance assembly 400 may be detachably-attachable to the cell expansion machine 202 (FIGS. 2 and 3) to facilitate relatively quick exchange of a new or unused pre-mounted fluid conveyance assembly 400 at a cell expansion machine 202 for a used pre-mounted fluid conveyance assembly 400 at the same cell expansion machine 202. As shown in FIG. 4, the bioreactor 100 may be attached to a bioreactor coupling that includes a shaft fitting 402. The shaft fitting 402 includes one or more shaft fastening mechanisms, such as a biased arm or spring member 404 for engaging a shaft, e.g., 258 (shown in FIG. 3), of the cell expansion machine 202.

According to embodiments, the pre-mounted fluid conveyance assembly 400 includes tubing 408A, 408B, 408C, 408D, 408E, etc., and various tubing fittings to provide the fluid paths shown in FIGS. 5 and 6, as discussed below. Pump loops 406A and 406B may also be provided for the pump(s). In embodiments, although the various media may be provided at the site where the cell expansion machine 202 is located, the pre-mounted fluid conveyance assembly 400 may include sufficient tubing length to extend to the exterior of the cell expansion machine 202 and to enable welded connections to tubing associated with media bag(s) or container(s), according to embodiments.

Next, FIG. 5 illustrates a schematic of an embodiment of a cell expansion system 500, and FIG. 6 illustrates a schematic of another embodiment of a cell expansion system 600. In the embodiments shown in FIGS. 5 and 6, and as described below, the cells are grown in the IC space. However, the disclosure is not limited to such examples and may in other embodiments provide for cells to be grown in the EC space.

FIG. 5 illustrates a CES 500, which includes first fluid circulation path 502 (also referred to as the “intracapillary loop” or “IC loop”) and second fluid circulation path 504 (also referred to as the “extracapillary loop” or “EC loop”), according to embodiments. First fluid flow path 506 may be fluidly associated with cell growth chamber 501 to form first fluid circulation path 502. Fluid flows into cell growth chamber 501 through IC inlet port 501A, through hollow fibers in cell growth chamber 501, and exits via IC outlet port 501B. Pressure gauge 510 measures the pressure of media leaving cell growth chamber 501. Media flows through IC circulation pump 512 which may be used to control the rate of media flow. IC circulation pump 512 may pump the fluid in a first direction or second direction opposite the first direction. Exit port 501B may be used as an inlet in the reverse direction. Media entering the IC loop may enter through valve 514. As those skilled in the art will appreciate, additional valves, pressure gauges, pressure/temperature sensors, ports, and/or other devices may be placed at various locations to isolate and/or measure characteristics of the media along portions of the fluid paths. Accordingly, it is to be understood that the schematic shown represents one possible configuration for various elements of the CES 500, and modifications to the schematic shown are within the scope of the one or more present embodiments.

With regard to the IC loop 502, samples of media may be obtained from sample port 516 or sample coil 518 during operation. Pressure/temperature gauge 520 disposed in first fluid circulation path 502 allows detection of media pressure and temperature during operation. Media then returns to IC inlet port 501A to complete fluid circulation path 502. Cells grown/expanded in cell growth chamber 501 may be flushed out of cell growth chamber 501 into harvest bag 599 through valve 598 or redistributed within the hollow fibers for further growth.

Fluid in second fluid circulation path 504 enters cell growth chamber 501 via EC inlet port 501C, and leaves cell growth chamber 501 via EC outlet port 501D. Media in the EC loop 504 may be in contact with the outside of the hollow fibers in the cell growth chamber 501, thereby allowing diffusion of small molecules into and out of the hollow fibers.

Pressure/temperature gauge 524 disposed in the second fluid circulation path 504 allows the pressure and temperature of media to be measured before the media enters the EC space of the cell growth chamber 501, according to an embodiment. Pressure gauge 526 allows the pressure of media in the second fluid circulation path 504 to be measured after it leaves the cell growth chamber 501. With regard to the EC loop, samples of media may be obtained from sample port 530 or a sample coil during operation.

In embodiments, after leaving EC outlet port 501D of cell growth chamber 501, fluid in second fluid circulation path 504 passes through EC circulation pump 528 to oxygenator or gas transfer module 532. EC circulation pump 528 may also pump the fluid in opposing directions. Second fluid flow path 522 may be fluidly associated with oxygenator or gas transfer module 532 via oxygenator inlet port 534 and oxygenator outlet port 536. In operation, fluid media flows into oxygenator or gas transfer module 532 via oxygenator inlet port 534, and exits oxygenator or gas transfer module 532 via oxygenator outlet port 536. Oxygenator or gas transfer module 532 adds oxygen to, and removes bubbles from, media in the CES 500, for example. In various embodiments, media in second fluid circulation path 504 may be in equilibrium with gas entering oxygenator or gas transfer module 532. The oxygenator or gas transfer module 532 may be any appropriately sized oxygenator or gas transfer device. Air or gas flows into oxygenator or gas transfer module 532 via filter 538 and out of oxygenator or gas transfer device 532 through filter 540. Filters 538 and 540 reduce or prevent contamination of oxygenator or gas transfer module 532 and associated media. Air or gas purged from the CES 500 during portions of a priming sequence may vent to the atmosphere via the oxygenator or gas transfer module 532.

In the configuration depicted for CES 500, fluid media in first fluid circulation path 502 and second fluid circulation path 504 flows through cell growth chamber 501 in the same direction (a co-current configuration). The CES 500 may also be configured to flow in a counter-current conformation.

In accordance with at least one embodiment, media, including cells (from bag 562), and fluid media from bag 546 may be introduced to first fluid circulation path 502 via first fluid flow path 506. Fluid container 562 (e.g., Cell Inlet Bag or Saline Priming Fluid for priming air out of the system) may be fluidly associated with the first fluid flow path 506 and the first fluid circulation path 502 via valve 564.

Fluid containers, or media bags, 544 (e.g., Reagent) and 546 (e.g., IC Media) may be fluidly associated with either first fluid inlet path 542 via valves 548 and 550, respectively, or second fluid inlet path 574 via valves 570 and 576. First and second sterile sealable input priming paths 508 and 509 are also provided. An air removal chamber (ARC) 556 may be fluidly associated with first circulation path 502. The air removal chamber 556 may include one or more ultrasonic sensors including an upper sensor and lower sensor to detect air, a lack of fluid, and/or a gas/fluid interface, e.g., an air/fluid interface, at certain measuring positions within the air removal chamber 556. For example, ultrasonic sensors may be used near the bottom and/or near the top of the air removal chamber 556 to detect air, fluid, and/or an air/fluid interface at these locations. Embodiments provide for the use of numerous other types of sensors without departing from the spirit and scope of the present disclosure. For example, optical sensors may be used in accordance with embodiments of the present disclosure. Air or gas purged from the CES 500 during portions of the priming sequence or other protocols may vent to the atmosphere out air valve 560 via line 558 that may be fluidly associated with air removal chamber 556.

EC media (e.g., from bag 568) or wash solution (e.g., from bag 566) may be added to either the first or second fluid flow paths. Fluid container 566 may be fluidly associated with valve 570 that may be fluidly associated with first fluid circulation path 502 via distribution valve 572 and first fluid inlet path 542. Alternatively, fluid container 566 may be fluidly associated with second fluid circulation path 504 via second fluid inlet path 574 and EC inlet path 584 by opening valve 570 and closing distribution valve 572. Likewise, fluid container 568 may be fluidly associated with valve 576 that may be fluidly associated with first fluid circulation path 502 via first fluid inlet path 542 and distribution valve 572. Alternatively, fluid container 568 may be fluidly associated with second fluid inlet path 574 by opening valve 576 and closing distribution valve 572.

An optional heat exchanger 552 may be provided for media reagent or wash solution introduction.

In the IC loop, fluid may be initially advanced by the IC inlet pump 554. In the EC loop, fluid may be initially advanced by the EC inlet pump 578. An air detector 580, such as an ultrasonic sensor, may also be associated with the EC inlet path 584.

In at least one embodiment, first and second fluid circulation paths 502 and 504 are connected to waste line 588. When valve 590 is opened, IC media may flow through waste line 588 and to waste or outlet bag 586. Likewise, when valve 582 is opened, EC media may flow through waste line 588 to waste or outlet bag 586.

In embodiments, cells may be harvested via cell harvest path 596. Here, cells from cell growth chamber 501 may be harvested by pumping the IC media containing the cells through cell harvest path 596 and valve 598 to cell harvest bag 599.

Various components of the CES 500 may be contained or housed within a machine or housing, such as cell expansion machine 202 (FIGS. 2 and 3), wherein the machine maintains cells and media, for example, at a predetermined temperature.

Turning to FIG. 6, a schematic of another embodiment of a cell expansion system 600 is shown. CES 600 includes a first fluid circulation path 602 (also referred to as the “intracapillary loop” or “IC loop”) and second fluid circulation path 604 (also referred to as the “extracapillary loop” or “EC loop”). First fluid flow path 606 may be fluidly associated with cell growth chamber 601 to form first fluid circulation path 602. Fluid flows into cell growth chamber 601 through IC inlet port 601A, through hollow fibers in cell growth chamber 601, and exits via IC outlet port 601B. Pressure sensor 610 measures the pressure of media leaving cell growth chamber 601. In addition to pressure, sensor 610 may, in embodiments, also be a temperature sensor that detects the media pressure and temperature during operation. Media flows through IC circulation pump 612 which may be used to control the rate of media flow. IC circulation pump 612 may pump the fluid in a first direction or second direction opposite the first direction. Exit port 601B may be used as an inlet in the reverse direction. Media entering the IC loop may enter through valve 614. As those skilled in the art will appreciate, additional valves, pressure gauges, pressure/temperature sensors, ports, and/or other devices may be placed at various locations to isolate and/or measure characteristics of the media along portions of the fluid paths. Accordingly, it is to be understood that the schematic shown represents one possible configuration for various elements of the CES 600, and modifications to the schematic shown are within the scope of the one or more present embodiments.

With regard to the IC loop, samples of media may be obtained from sample coil 618 during operation. Media then returns to IC inlet port 601A to complete fluid circulation path 602. Cells grown/expanded in cell growth chamber 601 may be flushed out of cell growth chamber 601 into harvest bag 699 through valve 698 and line 697. Alternatively, when valve 698 is closed, the cells may be redistributed within chamber 601 for further growth.

Fluid in second fluid circulation path 604 enters cell growth chamber 601 via EC inlet port 601C and leaves cell growth chamber 601 via EC outlet port 601D. Media in the EC loop may be in contact with the outside of the hollow fibers in the cell growth chamber 601, thereby allowing diffusion of small molecules into and out of the hollow fibers that may be within chamber 601, according to an embodiment.

Pressure/temperature sensor 624 disposed in the second fluid circulation path 604 allows the pressure and temperature of media to be measured before the media enters the EC space of the cell growth chamber 601. Sensor 626 allows the pressure and/or temperature of media in the second fluid circulation path 604 to be measured after it leaves the cell growth chamber 601. With regard to the EC loop, samples of media may be obtained from sample port 630 or a sample coil during operation.

After leaving EC outlet port 601D of cell growth chamber 601, fluid in second fluid circulation path 604 passes through EC circulation pump 628 to oxygenator or gas transfer module 632. EC circulation pump 628 may also pump the fluid in opposing directions, according to embodiments. Second fluid flow path 622 may be fluidly associated with oxygenator or gas transfer module 632 via an inlet port 632A and an outlet port 632B of oxygenator or gas transfer module 632. In operation, fluid media flows into oxygenator or gas transfer module 632 via inlet port 632A, and exits oxygenator or gas transfer module 632 via outlet port 632B. Oxygenator or gas transfer module 632 adds oxygen to, and removes bubbles from, media in the CES 600, for example. In various embodiments, media in second fluid circulation path 604 may be in equilibrium with gas entering oxygenator or gas transfer module 632. The oxygenator or gas transfer module 632 may be any appropriately sized device useful for oxygenation or gas transfer. Air or gas flows into oxygenator or gas transfer module 632 via filter 638 and out of oxygenator or gas transfer device 632 through filter 640. Filters 638 and 640 reduce or prevent contamination of oxygenator or gas transfer module 632 and associated media. Air or gas purged from the CES 600 during portions of a priming sequence may vent to the atmosphere via the oxygenator or gas transfer module 632.

In the configuration depicted for CES 600, fluid media in first fluid circulation path 602 and second fluid circulation path 604 flows through cell growth chamber 601 in the same direction (a co-current configuration). The CES 600 may also be configured to flow in a counter-current conformation, according to embodiments.

In accordance with at least one embodiment, media, including cells (from a source such as a cell container, e.g., a bag) may be attached at attachment point 662, and fluid media from a media source may be attached at attachment point 646. The cells and media may be introduced into first fluid circulation path 602 via first fluid flow path 606. Attachment point 662 may be fluidly associated with the first fluid flow path 606 via valve 664, and attachment point 646 may be fluidly associated with the first fluid flow path 606 via valve 650. A reagent source may be fluidly connected to point 644 and be associated with fluid inlet path 642 via valve 648, or second fluid inlet path 674 via valves 648 and 672.

Air removal chamber (ARC) 656 may be fluidly associated with first circulation path 602. The air removal chamber 656 may include one or more sensors including an upper sensor and lower sensor to detect air, a lack of fluid, and/or a gas/fluid interface, e.g., an air/fluid interface, at certain measuring positions within the air removal chamber 656. For example, ultrasonic sensors may be used near the bottom and/or near the top of the air removal chamber 656 to detect air, fluid, and/or an air/fluid interface at these locations. Embodiments provide for the use of numerous other types of sensors without departing from the spirit and scope of the present disclosure. For example, optical sensors may be used in accordance with embodiments of the present disclosure. Air or gas purged from the CES 600 during portions of a priming sequence or other protocol(s) may vent to the atmosphere out air valve 660 via line 658 that may be fluidly associated with air removal chamber 656.

An EC media source may be attached to EC media attachment point 668, and a wash solution source may be attached to wash solution attachment point 666, to add EC media and/or wash solution to either the first or second fluid flow path. Attachment point 666 may be fluidly associated with valve 670 that may be fluidly associated with first fluid circulation path 602 via valve 672 and first fluid inlet path 642. Alternatively, attachment point 666 may be fluidly associated with second fluid circulation path 604 via second fluid inlet path 674 and second fluid flow path 684 by opening valve 670 and closing valve 672. Likewise, attachment point 668 may be fluidly associated with valve 676 that may be fluidly associated with first fluid circulation path 602 via first fluid inlet path 642 and valve 672. Alternatively, fluid container 668 may be fluidly associated with second fluid inlet path 674 by opening valve 676 and closing distribution valve 672.

In the IC loop, fluid may be initially advanced by the IC inlet pump 654. In the EC loop, fluid may be initially advanced by the EC inlet pump 678. An air detector 680, such as an ultrasonic sensor, may also be associated with the EC inlet path 684.

In at least one embodiment, first and second fluid circulation paths 602 and 604 are connected to waste line 688. When valve 690 is opened, IC media may flow through waste line 688 and to waste or outlet bag 686. Likewise, when valve 692 is opened, EC media may flow to waste or outlet bag 686.

After cells have been grown in cell growth chamber 601, they may be harvested via cell harvest path 697. Here, cells from cell growth chamber 601 may be harvested by pumping the IC media containing the cells through cell harvest path 697, with valve 698 open, into cell harvest bag 699.

Various components of the CES 600 may be contained or housed within a machine or housing, such as cell expansion machine 202 (FIGS. 2 and 3), wherein the machine maintains cells and media, for example, at a predetermined temperature. It is further noted that, in embodiments, components of CES 600 and CES 500 (FIG. 5) may be combined. In other embodiments, a CES may include fewer or additional components than those shown in FIGS. 5 and 6 and still be within the scope of the present disclosure. An example of a cell expansion system that may incorporate features of the present disclosure is the Quantum® Cell Expansion System, manufactured by Terumo BCT, Inc. in Lakewood, Colo.

Examples and further description of cell expansion systems are provided in U.S. Pat. No. 8,309,347 (“Cell Expansion System and Methods of Use,” issued on Nov. 13, 2012), which is hereby incorporated by reference herein in its entirety for all that it teaches and for all purposes.

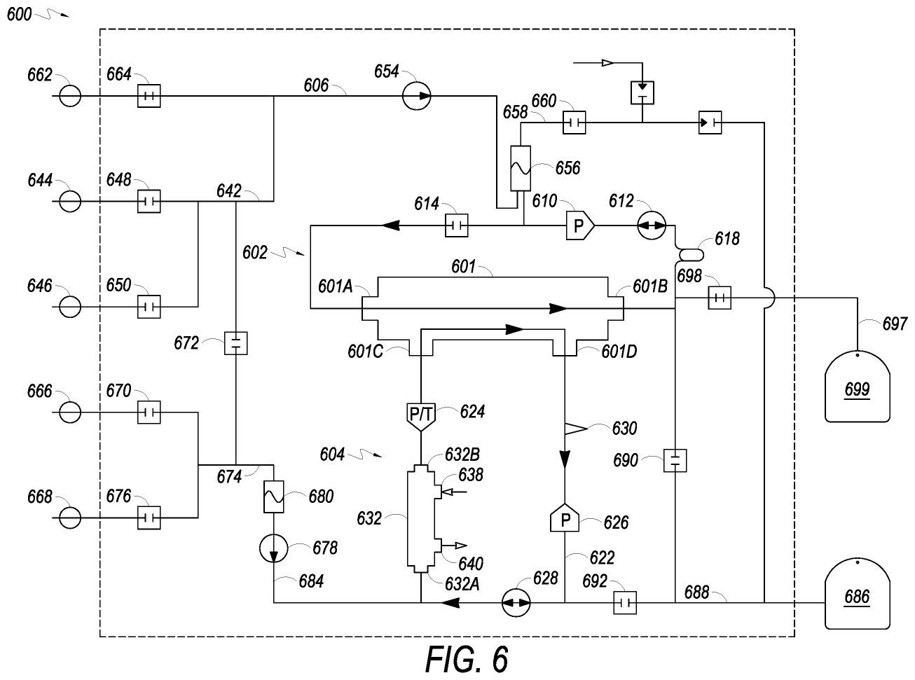

While various example embodiments of a cell expansion system and methods associated therewith have been described, FIG. 7 illustrates example operational steps 700 of a process for producing, purifying, and/or collecting released constituents, e.g., EVs or viral vectors, etc., that may be used with a cell expansion system, such as CES 500 (FIG. 5) or CES 600 (FIG. 6), in accordance with embodiments of the present disclosure. START operation is initiated 702, and process 700 proceeds to seed cells 704 in a bioreactor. In an embodiment, the cells are seeded on Day 0. In an embodiment, MSCs are seeded. Any cell type releasing a desired cellular product(s), e.g., EVs or viral vectors, etc., may be used as understood by those of skill in the art.

Next, the cells may be expanded 706 with media until confluent, according to an embodiment. In another embodiment, the cells may be expanded until a desired number of cell doublings occurs, e.g., one cell doubling, two cell doublings, three cell doublings, four cell doublings, five cell doublings, six or more cell doublings, etc. In another embodiment, the cells may be expanded for a particular time period. For example, the cells may be expanded for a time period of about twenty-four (24) hours to about forty-eight (48) hours. In another embodiment, the cells may be expanded for a time period of about forty-eight (48) hours to about seventy-two (72) hours. In another embodiment, the cells may be expanded for a period of time of about twenty-four (24) hours to about seventy-two (72) hours. In embodiments, such expansion occurs on Days 1 to 3, for example. According to another embodiment, the cells may be expanded for a time period of less than about twenty-four (24) hours. In another embodiment, the cells may be expanded for a time period of greater than seventy-two (72) hours. For example, in an embodiment, cells may be expanded for about seven (7) to about eight (8) days prior to collecting exosomes. Any period of time may be used in accordance with embodiments of the present disclosure.

The cells may be expanded 706 with complete media, for example. In an embodiment, the media comprises a serum-containing media, such as alpha-MEM (α-MEM) and a serum. In an embodiment, an animal-derived serum may be used. In another embodiment, a human-derived serum may be used. In another embodiment, a synthetic serum may be used. In yet another embodiment, another type of serum may be used. An example of a serum-containing media includes α-MEM+GlutaMAX+10% Fetal Bovine Serum (FBS). In a further embodiment, a serum-free media may be used. Any type of media known to those of skill in the art may be used.

Returning to FIG. 7, process 700 next proceeds to optional step 708 for washing out a first media, such as any serum-containing media, for example. In an embodiment, the serum-containing media is replaced with a second media, e.g., base media. An example of base media includes α-MEM+GlutaMAX. Any type of media known to those of skill in the art may be used. To isolate or purify the released constituents from the cells being expanded (as opposed to any also present in the serum or other protein source(s)), the washout procedure removes any serum, e.g., serum proteins, in accordance with embodiments. In an embodiment, the washout procedure occurs on Day 3. In an embodiment, step 708 may be optional where, for example, no animal-derived serum is used, only human-derived serum is used, serum-free media is used, and/or there are no other such additional protein sources to be removed, for example.

Next, process 700 proceeds to collect the released constituent(s) or agent(s) 710, e.g., EVs or viral vectors, etc., in the loop, e.g., IC loop. In an embodiment, such collection occurs by concentrating the released constituent(s) or agent(s), e.g., EVs or viral vectors, etc., in the IC loop by closing the IC outlet, e.g., closing the IC outlet valve. Such concentrating may occur for a defined time period. In an embodiment, such time period may include concentrating the released constituent(s) in the IC loop for about forty-eight (48) hours. In another embodiment, such time period may include concentrating the released constituent(s) for about twenty-four (24) hours to about forty-eight (48) hours. In another embodiment, such time period may include concentrating the released constituent(s) for about twenty-four (24) hours to about seventy-two (72) hours. In another embodiment, such time period may include concentrating the released constituent(s) for about forty-eight (48) hours to about seventy-two (72) hours. In an embodiment, such time period may include concentrating the released constituent(s) for greater than about seventy-two (72) hours. In an embodiment, such collection occurs on Days 3 to 5, for example. In yet another embodiment, such time period may include concentrating the released constituent(s) for less than about twenty-four (24) hours. Any time period may be used in accordance with embodiments of the present disclosure. During such collection period, the cells may be supplemented with media without protein, in which such media may be added from the EC side and diffused through the semi-permeable membrane. In another embodiment, media may be added from the IC side, in which the cells may be supplemented with media without protein, for example.

After collecting the released constituent(s) in the IC (or EC) loop, process 700 proceeds to harvest the released constituents 712. In an embodiment, such harvesting occurs on Day 5, for example. In an embodiment, released constituents, e.g., EVs or viral vectors, etc., may be transferred in suspension from the intracapillary circulation loop in the bioreactor to a harvest bag or harvest container. In an embodiment, media without protein may be used in such harvest task.

Next, process 700 optionally proceeds to further processing step 714, in which the harvested released constituent(s) may be processed for assays, for example. In another embodiment, such further processing 714 may include further concentrating of the released agent(s) from the media collected in the harvest bag(s). In another embodiment, such further processing 714 may include separating the released agent(s) from other components in the bag, such as cells where suspension cells may have been used and, thus, harvested with the released agent(s). In an embodiment, such further processing 714 may include further isolation and/or characterization of the released constituent(s). From optional further processing step 714, process 700 may terminate at END operation 716. Alternatively, process 700 may proceed directly to END operation 716 from harvest step 712 and terminate where there is no desire for optional further processing step 714.

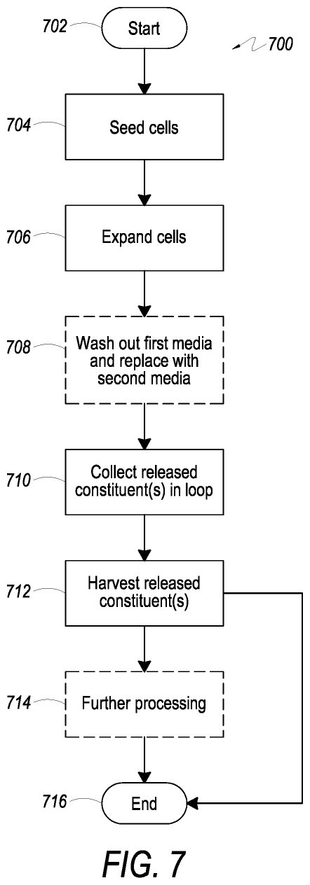

Next, FIG. 8 will be described in conjunction with example settings and media introduction. However, the embodiments presented herein are not limited to this example, but the embodiments can be modified to meet other requirements or system designs or configurations. START operation 802 is initiated, and process 800 proceeds to load the disposable tubing set 804 onto the cell expansion system.

Next, the system may be primed 806. In an embodiment, a user or an operator, for example, may provide an instruction to the system to prime by selecting a task for priming, for example. In an embodiment, such task for priming may be a pre-programmed task. The system 500 (FIG. 5) or 600 (FIG. 6) may be primed, for example, with Phosphate-buffered saline (PBS). To prime the bioreactor 501, 601, a bag (546) may be attached (for example, to connection point 646) to the system 500, 600. When referring to numerals in the Figures, for example, such as “Numeral, Numeral” (e.g., 500, 600), such nomenclature can mean “Numeral and/or Numeral” (e.g., 500 and/or 600). Valve 550, 650, may be opened. The PBS can then be directed into the first fluid circulation path 502, 602 by the IC inlet pump 554, 654 set to pump the PBS into the first fluid circulation path 502, 602. Valve 614 may be opened while the PBS enters the bioreactor 601 through the inlet 501A, 601A and out the outlet 501B, 601B. Once the bioreactor 501, 601 and/or the first fluid circulation path 502, 602 have media therein with air removed by the air removal chamber 556, 656, the bioreactor 501, 601 is primed, according to an embodiment.

To further prime the bioreactor 501, 601, a bag 586 may be attached (for example, to connection point 668) to the system 500, 600. Valve 576, 676 may be opened. The PBS can then be directed into the second fluid circulation path 502, 602 by the IC inlet pump 554, 654 set to pump the PBS into the first fluid circulation path 504, 604. Valve 692 may be closed while the PBS enters the bioreactor 601 through the inlet 501C, 601C and out the outlet 501D, 601D of the EC loop. Once the bioreactor 501, 601 and/or the second fluid circulation path 504, 604 have media therein with air removed by the air removal chamber 580, 680, the bioreactor 501, 601 is primed, according to an embodiment.

Process 800 then proceeds to coat the bioreactor 808, in which the bioreactor 501, 601 may be coated with a coating agent, for example, 5 mg of Fibronectin (FN). For example, in embodiments, a reagent bag 544 may be loaded (for example, on connection point 644) into an IC loop 502, 602 until a reagent container 544 is empty. The reagent 544 may be chased from an air removal chamber 556, 656 into the IC loop 502, 602, and the reagent 544 may then be circulated in the IC loop 502, 602 by operating the circulation pump 512, 612 and/or the inlet pump 554, 654. Any coating reagent known to those of skill in the art may be used, such as FN or cryoprecipitate, for example.

An example of the solutions being introduced to the system 500, 600 to coat the bioreactor may be as shown below:

TABLE 1

| Bag | Solution | Volume (estimation based on factory |
|---|---|---|
| (Connection Point) | in Bag | default values) |
| Cell Inlet 562 (662) | None | N/A |
| Reagent 544 (644) | Fibronectin | 5 mg Fibronectin in 100 mL PBS |
| IC Media 546 (646) | None | N/A |
| Wash 566 (666) | PBS | 0.1 L + 6 mL/hr (overnight) |
| EC Media 568 (668) | None | N/A |

The coating of the bioreactor may occur in three stages. An example of the settings for the system 500, 600 for the first stage of introducing the solutions above may be as shown below:

TABLE 2

| Component | Setting |
|---|---|
| IC Inlet valve configuration | Reagent (valve 548, 648 open, |
| | other valves closed) |
| IC Inlet Rate for Pump 554, 654 | 10 mL/min |
| IC Circulation Rate for Pump 512, 612 | 100 mL/min |
| EC Inlet valve configuration | None (valves closed) |
| EC Inlet Rate for Pump 578, 678 | 0 mL/min |
| EC Circulation Rate for Pump 528, 628 | 30 mL/min |
| Outlet valve configuration | EC Outlet (valve 582 open) |
| Rocker Control | Stationary (0°) |
| Stop Condition | Empty Bag for bag 544 |

An example of the settings for the system 500, 600 for the second stage of coating the bioreactor, which chases reagent from the air removal chamber 556, 656, may be as shown below:

TABLE 3

| Component | Setting |
|---|---|
| IC Inlet valve configuration | Wash (valve 560, 660 open, |
| | other valves closed) |
| IC Inlet Rate for Pump 554, 654 | 10 mL/min |
| IC Circulation Rate for Pump 512, 612 | 100 mL/min |
| EC Inlet valve configuration | None (valves closed) |
| EC Inlet Rate for Pump 578, 678 | 0 mL/min |
| EC Circulation Rate for Pump 528, 628 | 30 mL/min |
| Outlet valve configuration | EC Outlet (valve 582 open) |
| Rocker Control | Stationary (0°) |
| Stop Condition | IC Volume (e.g., 22 mL) |

An example of the settings for the system 500, 600 for the third stage of coating the bioreactor, which circulates reagent in the IC loop 502, 602, may be as shown below:

TABLE 4

| Component | Setting |
|---|---|
| IC Inlet valve configuration | None (valves closed) |
| IC Inlet Rate for Pump 554, 654 | 0 mL/min |
| IC Circulation Rate for Pump 512, 612 | 20 mL/min |
| EC Inlet valve configuration | Wash (valves 576, 676 open) |
| EC Inlet Rate for Pump 578, 678 | 0.1 mL/min |
| EC Circulation Rate for Pump 528, 628 | 30 mL/min |
| Outlet valve configuration | EC Outlet (valve 582, 692 open) |
| Rocker Control | Stationary (0°) |
| Stop Condition | Manual |

Once the bioreactor is coated, the IC/EC Washout task may be performed in step 810, in which fluid on the IC circulation loop 502, 602 and on the EC circulation loop 504, 604 may be replaced. The replacement volume may be determined by the number of IC Volumes and EC Volumes exchanged. An example of the solutions being introduced to the CES 500, 600 during the IC/EC Washout task may be as shown below:

TABLE 5

| Bag | | Volume (estimation based |
|---|---|---|
| (Connection Point) | Solution in Bag | on factory default values) |
| Cell Inlet 562 (662) | None | N/A |
| Reagent 544 (644) | None | N/A |
| IC Media 546 (646) | Media with Protein | 1.4 L |
| Wash 566 (666) | None | N/A |
| EC Media 568 (668) | None | N/A |

An example of the settings for an IC/EC Washout task of the system 500, 600 may be as shown below:

TABLE 6

| Component | Setting |
|---|---|
| IC Inlet valve configuration | IC Media (e.g., Serum with |
| | Protein) |
| IC Inlet Rate for Pump 554, 654 | 100 mL/min |
| IC Circulation Rate for Pump 512, 612 | −17 mL/min |
| EC Inlet valve configuration | IC Media (valve 550, 650 open, |
| | other valves closed) |
| EC Inlet Rate for Pump 578, 678 | 148 mL/min |
| EC Circulation Rate for Pump 528, 628 | −1.7 mL/min |
| Outlet valve configuration | IC and EC Outlet (valves 590, |
| | 690 and 582, 692 open) |
| Rocker Control | In Motion (−90°, 180°, |
| | in 1 sec intervals) |
| Stop Condition | Exchange (2.5 IC Volumes; |
| | 2.5 EC Volumes) |

Next, to maintain the proper or desired gas concentration across the fibers in the bioreactor membrane, the condition media task 812 may be executed to allow the media to reach equilibrium with the provided gas supply before cells are loaded into the bioreactor. For example, rapid contact between the media and the gas supply provided by the gas transfer module or oxygenator 532, 632 is provided by using a high EC circulation rate. The system 500, 600 may then be maintained in a proper or desired state until a user or operator, for example, is ready to load cells into the bioreactor 501, 601. In an embodiment, the CES 500, 600 may be conditioned with complete media, for example. Complete media may be any media source used for cell growth. In an embodiment, complete media may comprise alpha-MEM (α-MEM) and fetal bovine serum (FBS), for example. Any type of media known to those of skill in the art may be used.

The condition media task 812 may be a two-step process where, in the first step, the system 500, 600 provides rapid contact between the media and the gas supply by using a high EC circulation rate. In the second step, the system 500, 600 maintains the bioreactor 501, 601 in a proper state until the operator is ready to load the cells. An example of the solutions being introduced to the CES 500, 600 during the condition media task 812 may be as shown below:

TABLE 7

| Bag | | Volume (estimation based |
|---|---|---|
| (Connection Point) | Solution in Bag | on factory default values) |
| Cell Inlet 562 (662) | None | N/A |
| Reagent 544 (644) | None | N/A |
| IC Media 546 (646) | Media with Protein | 0.1 L plus 6 mL/hour |
| | (e.g., αMEM with | |
| | GlutaMAX plus | |
| | 10% FBS) | |
| Wash 566 (666) | None | N/A |
| EC Media 568 (668) | None | N/A |

An example of the settings for a first step of the condition media task 812 may be as shown below:

TABLE 8

| Component | Setting |
|---|---|
| IC Inlet valve configuration | None |
| IC Inlet Rate for Pump 554, 654 | 0 mL/min |
| IC Circulation Rate for Pump 512, 612 | 100 mL/min |
| EC Inlet valve configuration | IC Media (valve 550, 650 |
| | open, other valves closed) |
| EC Inlet Rate for Pump 578, 678 | 0.1 mL/min |
| EC Circulation Rate for Pump 528, 628 | 250 mL/min |
| Outlet valve configuration | EC Outlet (valves 582, 692 |
| | open) |
| Rocker Control | Stationary |
| Stop Condition | Time (e.g., 10 min) |

An example of the settings for a second step of the condition media task 812 may be as shown below:

TABLE 9

| Component | Setting |
|---|---|
| IC Inlet valve configuration | None |
| IC Inlet Rate for Pump 554, 654 | 0 mL/min |
| IC Circulation Rate for Pump 512, 612 | 100 mL/min |
| EC Inlet valve configuration | IC Media (valve 550, 650 open, |
| | other valves closed) |
| EC Inlet Rate for Pump 578, 678 | 0.1 mL/min |
| EC Circulation Rate for Pump 528, 628 | 30 mL/min |
| Outlet valve configuration | EC Outlet (valves 582, 692 |
| | open) |
| Rocker Control | Stationary |
| Stop Condition | Manual |

Process 800 next proceeds to loading cells 814 into the bioreactor 501, 601 from a cell inlet bag 562 (at connection point 662), for example. Loading cells can be a three step process. First, the cells can be loaded, with a uniform suspension 814, for example, into the bioreactor 501, 601 from the cell inlet bag 562 (at connection point 662) until the bag 562 is empty. In another embodiment, the cells may be loaded 814 by another type of loading procedure, such as through a bulls-eye loading procedure, for example. Any type of loading procedure may be used in accordance with embodiments. Second, cells may then be chased from the air removal chamber 556, 656 to the bioreactor 501, 601. In configurations that utilize larger chase volumes, cells may be spread and move toward the IC outlet 590, 690. In a third step, the distribution of cells may be promoted across the membrane via IC circulation, such as through an IC circulation pump 514, 614, with no IC inlet, for example.

An example of the solutions being introduced to the system 500, 600 to load cells 814 may be as shown below:

TABLE 10

| Bag | Solution | Volume (estimation based |
|---|---|---|
| (Connection Point) | in Bag | on factory default values) |
| Cell Inlet 562 (662) | Cells | Cells (e.g., mesenchymal stem cells |
| | | (MSC)) in 100 mL complete media |
| Reagent 544 (644) | None | N/A |
| IC Media 546 (646) | Media with | 0.1 L |
| | Protein | |
| Wash 566 (666) | None | N/A |
| EC Media 568 (668) | None | N/A |

As explained above, the loading cells 814 may occur in three stages. An example of the settings for the system 500, 600 for the first stage may be as shown below:

TABLE 11

| Component | Setting |
|---|---|
| IC Inlet valve configuration | Cell Inlet (valve 564, 664 open, |
| | other valves closed) |
| IC Inlet Rate for Pump 554, 654 | 50 mL/min |
| IC Circulation Rate for Pump 512, 612 | 200 mL/min |
| EC Inlet valve configuration | None (valves closed) |
| EC Inlet Rate for Pump 578, 678 | 0 mL/min |
| EC Circulation Rate for Pump 528, 628 | 30 mL/min |
| Outlet valve configuration | EC Outlet (valve 582 open) |
| Rocker Control | In Motion (−90°, 180°, |
| | in 1 sec intervals) |
| Stop Condition | ARC stop |

An example of the settings for the system 500, 600 for the second stage may be as shown below:

TABLE 12

| Component | Setting |
|---|---|
| IC Inlet valve configuration | IC Media (valve 550, 650 open, |
| | other valves closed) |
| IC Inlet Rate for Pump 554, 654 | 50 mL/min |
| IC Circulation Rate for Pump 512, 612 | 200 mL/min |
| EC Inlet valve configuration | None (valves closed) |
| EC Inlet Rate for Pump 578, 678 | 0 mL/min |
| EC Circulation Rate for Pump 528, 628 | 30 mL/min |
| Outlet valve configuration | EC Outlet (valve 582 open) |
| Rocker Control | In Motion (−90°, 180°, |
| | in 1 sec intervals) |
| Stop Condition | IC Volume (e.g., 22 mL) |

An example of the settings for the system 500, 600 for the third stage may be as shown below:

TABLE 13

| Component | Setting |
|---|---|
| IC Inlet valve configuration | None (valves closed) |
| IC Inlet Rate for Pump 554, 654 | 0 mL/min |
| IC Circulation Rate for Pump | 20 mL/min |
| 512, 612 | |
| EC Inlet valve configuration | None |
| EC Inlet Rate for Pump 578, 678 | 0 mL/min |
| EC Circulation Rate for Pump | 30 mL/min |
| 528, 628 | |
| Outlet valve configuration | EC Outlet (valve 582, 692 open) |
| Rocker Control | In Motion (−90°, 180°, in |
| | 1 sec intervals) |
| Stop Condition | Time (2.0 Min) |

Further, the cells, e.g., adherent cells, may be allowed to attach 816 to the hollow fibers, for example. In an embodiment, in allowing the cells to attach 816, adherent cells are enabled to attach to the bioreactor membrane while allowing flow on the EC circulation loop 504, 604, with the pump 514, 614 flow rate to the IC loop 502, 602 set to zero. An example of the solutions being introduced to the CES 500, 600 during the process of cells attaching to the membrane 816 may be as shown below:

TABLE 14

| | | Volume |
|---|---|---|
| | | (estimation based on |
| Bag (Connection Point) | Solution in Bag | factory default values) |
| Cell Inlet 562 (662) | None | N/A |
| Reagent 544 (644) | None | N/A |
| IC Media 546 (646) | Media with | 6 mL/hour |
| | Protein | |
| Wash 566 (666) | None | N/A |
| EC Media 568 (668) | None | N/A |

An example of the settings for attaching to the membrane 816 in the system 500, 600 may be as shown below:

TABLE 15

| Component | Setting | |
|---|---|---|
| IC Inlet valve configuration | None | |
| IC Inlet Rate for Pump 554, 654 | 0 | mL/min |
| IC Circulation Rate for Pump | 0 | mL/min |
| 512, 612 | | |
| EC Inlet valve configuration | IC Media (valve 550, 650 open, | |
| | other valves closed) | |
| EC Inlet Rate for Pump 578, 678 | 0.1 | mL/min |
| EC Circulation Rate for Pump | 30 | mL/min |
| 528, 628 | | |

| | |
|---|---|
| Outlet valve configuration | EC Outlet (valve 582, 692 open) |
| Rocker Control | Stationary (at 180°) |
| Stop Condition | Manual |

The cells may be fed in step 818, in which a flow rate, e.g., a low flow rate, may be continuously added to the IC circulation loop 502, 602 and/or the EC circulation loop 504, 604. Outlet settings allow for the removal of fluid added to the system 500, 600. An example of the solutions being introduced to the system 500, 600 during the feed step 818 may be as shown below:

TABLE 16

| | | Volume |
|---|---|---|
| | | (estimation based on |
| Bag (Connection Point) | Solution in Bag | factory default values) |
| Cell Inlet 562 (662) | None | N/A |
| Reagent 544 (644) | None | N/A |
| IC Media 546 (646) | Media with | 6 mL/hour |
| | Protein | |
| Wash 566 (666) | None | N/A |
| EC Media 568 (668) | None | N/A |

An example of the settings for the feed step 818 in the system 500, 600 may be as shown below:

TABLE 17

| Component | Setting |
|---|---|
| IC Inlet valve configuration | None |
| IC Inlet Rate for Pump 554, 654 | 0.1 mL/min |
| IC Circulation Rate for Pump | 20 mL/min |
| 512, 612 | |
| EC Inlet valve configuration | None |
| EC Inlet Rate for Pump 578, 678 | 0 mL/min |
| EC Circulation Rate for Pump | 30 mL/min |
| 528, 628 | |
| Outlet valve configuration | IC Outlet (valves 590, 690 open) |
| Rocker Control | Stationary (at 0°) |
| Stop Condition | Manual |

Next, process 800 can proceed to an optional step 820 to wash out any serum-containing media and replace with base media or media without protein. If the previous processing uses media without protein to load the cells, feed the cells, etc., then step 820 may not be needed. However, if serum-containing protein is used, a washout procedure 820 may be initiated in anticipation of isolating and/or collecting released cellular product(s), e.g., EVs or viral vectors, etc., after the cells have reached confluence, after a defined period of time, or after a number of desired cell doublings is reached, for example.

For example, it may be desired to initiate the purification and/or collection of released cellular product(s) after a minimum of two cell doublings, three cell doublings, four cell doublings, five cell doublings, six or more cell doublings, etc. One or more processes may be used to purify the media in the system 500, 600 for collection of the released EV products. Such purification procedures may comprise a 5× IC EC Washout 820, a Negative Ultrafiltration Washout 822, an IC EC Washout 824, and/or another type of washout procedure. First, the 5× X IC EC Washout 820 may include replacing the fluid on both the IC circulation loop 502, 602 and the EC circulation loop 504, 604, in which the replacement volume may be specified by the number of IC volumes and EC volumes exchanged.

An example of the solutions being introduced to the system 500, 600 during the 5× IC/EC Washout task 820 may be as shown below:

TABLE 18

| | | Volume |
|---|---|---|
| | | (estimation based on |
| Bag (Connection Point) | Solution in Bag | factory default values) |
| Cell Inlet 562 (662) | None | N/A |
| Reagent 544 (644) | None | N/A |
| IC Media 546 (646) | None | N/A |
| Wash 566 (666) | PBS | 2.8 L |
| EC Media 568 (668) | None | N/A |

An example of the settings for 5× IC/EC Washout step 820 of the system 500, 600 may be as shown below:

TABLE 19

| Component | Setting |
|---|---|
| IC Inlet valve configuration | Wash (valve 570, 670 and 572, 672 |
| | open, other valves closed) |
| IC Inlet Rate for Pump 554, 654 | 100 mL/min |
| IC Circulation Rate for Pump | −17 mL/min |
| 512, 612 | |
| EC Inlet valve configuration | Wash (valve 570, 670 open) |
| EC Inlet Rate for Pump 578, 678 | 148 mL/min |
| EC Circulation Rate for Pump | −1.7 mL/min |
| 528, 628 | |
| Outlet valve configuration | IC and EC Outlet (valves 590, |
| | 690 and 582, 692 open) |
| Rocker Control | In Motion (−90°, 180°, |
| | in 1 sec intervals) |
| Stop Condition | Exchange (5.0 IC Volumes; |
| | 5.0 EC Volumes) |

Further, an optional Negative Ultrafiltration Washout 822 may comprise washing the IC circulation loop 502, 602 using negative ultrafiltration to help lift off any constituents that may have settled on the IC side of the fibers. An example of the solutions being introduced to the CES 500, 600 during the negative ultrafiltration 822 may be as shown below:

TABLE 20

| | | Volume |
|---|---|---|
| | | (estimation based on |
| Bag (Connection Point) | Solution in Bag | factory default values) |
| Cell Inlet 562 (662) | None | N/A |
| Reagent 544 (644) | None | N/A |
| IC Media 546 (646) | None | N/A |
| Wash 566 (666) | PBS | 400 mL |
| EC Media 568 (668) | None | N/A |

An example of the settings for negative ultrafiltration 822 of the system 500, 600 may be as shown below:

TABLE 21

| Component | Setting |
|---|---|
| IC Inlet valve configuration | Wash (valve 570, 670 and 572, 672 |
| | open, other valves closed) |
| IC Inlet Rate for Pump 554, 654 | 200 mL/min |
| IC Circulation Rate for Pump | −69 mL/min |
| 512, 612 | |
| EC Inlet valve configuration | Wash (valve 570, 670 open) |
| EC Inlet Rate for Pump 578, 678 | 60 mL/min |
| EC Circulation Rate for Pump | 30 mL/min |
| 528, 628 | |
| Outlet valve configuration | IC and EC Outlet (valves 590, |
| | 690 and 582, 692 open) |
| Rocker Control | In Motion (−90°, 180°, |
| | in 1 sec intervals) |
| Stop Condition | IC Volume (400 mL) |

Further, an optional IC EC Washout 824 may be used to replace the fluid on both the IC circulation loop 502, 602 and the EC circulation loop 504, 604, in which the replacement volume may be specified by the number of IC volumes and EC volumes exchanged. In such washout procedure(s), the media used may be base media or media without protein, for example. An example of the solutions being introduced to the system 500, 600 during the optional IC EC Washout 824 may be as shown below:

TABLE 22

| | | Volume |
|---|---|---|
| | | (estimation based on |
| Bag (Connection Point) | Solution in Bag | factory default values) |
| Cell Inlet 562 (662) | None | N/A |
| Reagent 544 (644) | None | N/A |
| IC Media 546 (646) | None | N/A |
| Wash 566 (666) | None | N/A |
| EC Media 568 (668) | Media without | 1.4 L |
| | Protein | |

An example of the settings for IC EC Washout 824 of the system 500, 600 may be as shown below:

TABLE 23

| Component | Setting | |
|---|---|---|
| IC Inlet valve configuration | EC Media (valves 576, 676 and 572, | |
| | 672 open, other valves closed) | |
| IC Inlet Rate for Pump 554, 654 | 100 | mL/min |
| IC Circulation Rate for Pump | −17 | mL/min |
| 512, 612 | | |
| EC Inlet valve configuration | EC Media (valve 576, 676 open) | |
| EC Inlet Rate for Pump 578, 678 | 148 | mL/min |
| EC Circulation Rate for Pump | −1.7 | mL/min |
| 528, 628 | | |

| | |
|---|---|
| Outlet valve configuration | IC and EC Outlet (valves 590, |
| | 690 and 582, 692 open) |
| Rocker Control | In Motion (−90°, 180°, |
| | in 1 sec intervals) |
| Stop Condition | Exchange (2.5 IC Volumes; |
| | 2.5 EC volumes) |

Such tasks described above may be custom or user-defined tasks. In other configurations, such tasks may be pre-programmed or default tasks. In other embodiments, such tasks may be performed manually by a user or operator, for example.

In embodiments, the total protein in the system 500, 600 may be measured on the IC side (or EC side, for example, in other configurations at different points during the washout procedure, e.g., before 5× washout, after 5× washout 820, after negative ultrafiltration washout 822, after base media exchange 824, to demonstrate removal, e.g., complete removal, of serum or serum protein(s). Further, steps 820, 822, and/or 824 may be optional where, for example, no animal-derived serum is used, only human-derived serum is used, serum-free media is used, and/or there are no other such additional protein sources to be removed, for example.

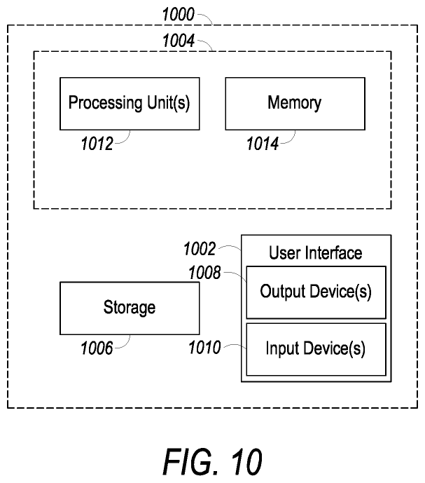

The efficacy of the above methods may be represented by the graph 1100 in FIG. 11, according to an embodiment. Graph 1100 represents possible results of measurements of protein in a system, such as system 500, 600, implementing the procedures discussed above. As shown, the amount of protein in the system 500, 600, may be represented by line 1104. Before the above procedures 820, 822, 824, the amount of protein in the system 500, 600 may be at a peak 1108 (e.g., over 7000 μg/mL). During the 5× washout 820, the amount of protein may drop steadily, as represented by portion 1112 of line 1104. Indeed, at the end of the 5× washout 820, the protein concentration may be substantially 0 μg/mL, as represented by portion 1116 of line 1104. In such cases, there may be no need for negative ultrafiltration washout 822 or base media exchange 824, for example. However, procedures 822 and 824 may further effectively extract protein in the system 500, 600, according to an embodiment.

Following the washout of any serum-containing media from the bioreactor, the cells in the bioreactor may be fed with media without protein 826 for a defined period of time, e.g., about forty-eight (48) hours, through media added to the EC side 504, 604 of the bioreactor 502, 602 and diffusion through the semi-permeable membrane. The EC inlet 668 can supply EC media, e.g., media without protein, for providing nutrients to the cells while the released cellular product(s) are being allowed to concentrate. In such configurations, there may be no IC inlet. Instead, the semi-permeable hollow fibers of the bioreactor 502, 602 allow essential nutrients (e.g., glucose) to reach the cells by continuous perfusion, and metabolic waste products (e.g., lactate) may be actively removed and may exit the system 500, 600 via diffusion (EC outlet open 582, 692). Such feeding may occur for a defined time period, e.g., about forty-eight (48) hours. In another situation, media without protein is used for about twenty-four (24) hours to about seventy-two (72) hours to supplement the cells. In another embodiment, such feeding with media without protein, e.g., a second media, occurs for about forty-eight (48) hours to about seventy-two (72) hours to supplement the cells. In another embodiment, such feeding with a second media, e.g., media without protein, occurs for about twenty-four (24) hours to about forty-eight (48) hours. In another embodiment, such feeding with a second media occurs for about twenty-four (24) hours to about forty-eight (48) hours. In yet another embodiment, such feeding with media without protein occurs for less than about twenty-four (24) hours. Any time period of feeding with a second media may be used in accordance with embodiments. In another configuration, the IC inlet can supply IC media, e.g., media without protein, for providing nutrients to the cells. Such feeding may occur for any time period in accordance with embodiments of the present disclosure. Such feeding of the cells 826 may include the closing of the IC outlet by closing the IC outlet valve 590, 690. An example of the solutions being introduced to the system 500, 600 during the feeding of the cells 826 may be as shown below:

TABLE 24

| | | Volume |
|---|---|---|
| | | (estimation based on |
| Bag (Connection Point) | Solution in Bag | factory default values) |
| Cell Inlet 562 (662) | None | N/A |
| Reagent 544 (644) | None | N/A |
| IC Media 546 (646) | None (optionally | N/A (optionally 0.1 |
| | Media without | mL/min, for example) |
| | protein) | |
| Wash 566 (666) | None | N/A |
| EC Media 568 (668) | Media without | 0.1 mL/min |
| | Protein | for 48 hours |

An example of the settings for feeding of the cells 826 with the system 500, 600 may be as shown below:

TABLE 25

| Component | Setting |
|---|---|
| IC Inlet valve configuration | None (optionally valve 650 |
| | open to feed from bag 546) |
| IC Inlet Rate for Pump 554, 654 | 0.0 mL/min (optionally |
| | 0.1 mL/min feed rate) |
| IC Circulation Rate for Pump | 20 mL/min |
| 512, 612 | |
| EC Inlet valve configuration | EC Media (valve 576, 676 open) |
| EC Inlet Rate for Pump 578, 678 | 0.1 mL/min |
| EC Circulation Rate for Pump | 30 mL/min |
| 528, 628 | |
| Outlet valve configuration | EC Outlet (valves 582, 692 open) |
| Rocker Control | Stationary (0°) |
| Stop Condition | Manual |

In another embodiment, the closing of the IC outlet to allow for the collection of released cellular product(s) occurs in step 828, for example. Such closing of the IC outlet (by closing valve 590, 598 or 690, 698) allows for cellular product(s) released by the cells to collect or concentrate in the bioreactor 828. By keeping the EC outlet open (valves 582, 692), ultrafiltration allows for the active removal of waste via the EC side 504, 604. Further, the semi-permeable membrane allow essential nutrients to reach the cells by continuous perfusion. As noted in Tables 24 and 25, for example, the cells may be fed 826 by optionally adding media (e.g., without protein) on the IC side, according to an embodiment. In an embodiment, media bag 546 may be connected to connection point 646. Media (e.g., without protein) from bag 546 may be sent through valve 550, 650 into IC inlet line 506, 606. From the inlet line 506, 606, media can enter the IC loop 502, 602 to feed the cells in the IC portion of the bioreactor 501, 601.

Additionally or alternatively, step(s) 826 and/or 828 may also include the optional replacement 827, 829 of the outlet or waste bag(s) 586, 686. Such replacement of the outlet or waste bag(s) 586, 686 allows for monitoring or testing of the bag's contents to be performed to monitor glucose/lactate amounts, to determine if any released cellular product(s) are crossing the membrane (since the IC outlet is closed), etc., in an embodiment. As noted above, cellular product(s), e.g., EVs or viral vectors, etc., produced by the cells may be too large to diffuse through the membrane, e.g., their molecular weights are too large. Where the IC outlet is closed, the released cellular product(s), e.g., EVs, may be maintained on the IC side of the bioreactor 501, 601 during expansion (or defined collection period) where the released cellular product, e.g., EV, concentration is continuously increased. The feeding of the cells 826 can occur simultaneously with the closing of the IC outlet. In another embodiment, step 826 occurs after closing the IC outlet at collect step 828, for example. In yet another embodiment, step 826 occurs prior to closing the IC outlet at collect step 828, for example. In other embodiments, the outlet bag 586, 686 is not replaced where no testing/monitoring is desired after the closing of the IC outlet. In yet another embodiment, the outlet or waste container(s) or bag(s) may be used for collecting and harvesting released agent(s). While the IC outlet is referred to as being closed in process 800, it should be noted that the EC outlet may be closed in other embodiments where cell growth may occur on the EC side, for example.

When it is determined to begin collecting the concentrated or collected cellular product(s) 828 in the loop, e.g., IC loop 502, 602, (such as after about forty-eight (48) hours, or another time period, for example), the valve(s) 598, 698 for harvest is opened 830, and the cellular product(s) can be harvested 830 from the IC side 502, 602 of the bioreactor 501, 601 to a harvest container(s) 599, 699, according to an embodiment. In an embodiment, the IC valve 590, 690 for the IC outlet 586, 686 is opened to allow for harvesting. In another embodiment, a harvest valve 598, 698 is opened to allow for harvesting to the harvest bag(s) 599, 699 or harvest container(s). In an embodiment, such harvesting occurs at the end of the entire process. In another embodiment, such harvesting occurs at defined interval(s). In an embodiment, cellular product(s), e.g., EVs or viral vectors, etc., may be transferred in suspension from the intracapillary circulation loop 502, 602 in the bioreactor 501, 601 to a harvest bag 599, 699 or harvest container. In an embodiment, media without protein may be used in such harvest task. An example of the solutions being introduced to the system 500, 600 during the collection of the concentrated or collected cellular product(s) 828 may be as shown below:

TABLE 26

| | | Volume |
|---|---|---|
| | | (estimation based on |
| Bag (Connection Point) | Solution in Bag | factory default values) |
| Cell Inlet 562 (662) | None | N/A |
| Reagent 544 (644) | None | N/A |
| IC Media 546 (646) | None | N/A |
| Wash 566 (666) | None | N/A |
| EC Media 568 (668) | Media without | 0.5 L |
| | Protein | |

An example of the settings for the collection of the concentrated or collected cellular product(s) 828 with the system 500, 600 may be as shown below:

TABLE 27

| Component | Setting | |
|---|---|---|
| IC Inlet valve configuration | EC Media (valves 572, 672 open; | |
| | other valves closed) | |
| IC Inlet Rate for Pump 554, 654 | 20 | mL/min |
| IC Circulation Rate for Pump | 2 | mL/min |
| 512, 612 | | |
| EC Inlet valve configuration | None | |
| EC Inlet Rate for Pump 578, 678 | 0 | mL/min |
| EC Circulation Rate for Pump | 30 | mL/min |
| 528, 628 | | |
| Outlet valve configuration | Harvest (valves 598, 698 open) | |
| Rocker Control | Stationary (−90°) | |
| Stop Condition | 150 | mL |

Following the harvesting of the released agent(s) 830, process 800 may proceed directly from harvest cellular product(s) 830 to terminate at END operation 838 where no further processing 832 or harvesting of the cells 836 is desired.

Alternatively, from harvest agent(s) 830, process 800 may optionally proceed to allow for further processing 832. Such further processing 832 may include processing for assay(s), in an embodiment. In another embodiment, such further processing 832 may include further concentrating of the cellular product(s) from the media or fluid collected in the harvest bag(s). In another embodiment, such further processing 832 may include separating the cellular product(s) from other components in the bag, such as cells where suspension cells may be expanded and harvested with the released product(s). In an embodiment, such further processing 832 may include further isolating and/or characterization of the released cellular product(s). From optional further processing step 832, process 800 may terminate at END operation 838 where there is no desire to harvest cells, e.g., any adherent cells, remaining in the bioreactor.

On the other hand, if it is desired to harvest the cells, e.g., any adherent or attached cells, remaining in the bioreactor, process 800 proceeds to release cells 834. Alternatively, process 800 may proceed directly to release cells 834 from harvest cellular product(s) 830 where no further processing 832 is desired, and it is desired to release cells 834 from the bioreactor. Attached cells may be released 834 from the membrane of the bioreactor and suspended in the IC loop, for example. In embodiments, an IC/EC washout task in preparation for adding a reagent to release the cells is performed as part of operation 834. For example, IC/EC media may be replaced with a phosphate buffered saline (PBS) to remove protein, calcium (Ca²⁺), and magnesium (Mg²⁺) in preparation for adding trypsin, or another chemical-releasing agent, to release any adherent cells. A reagent may be loaded into the system until the reagent bag is empty. The reagent may be chased into the IC loop, and the reagent may be mixed within the IC loop. Following the release of any adherent cells, harvest operation 836 transfers the cells in suspension from the IC circulation loop, for example, including any cells remaining in the bioreactor, to a harvest bag(s) or container(s). Finally, process 800 then terminates at END operation 838.

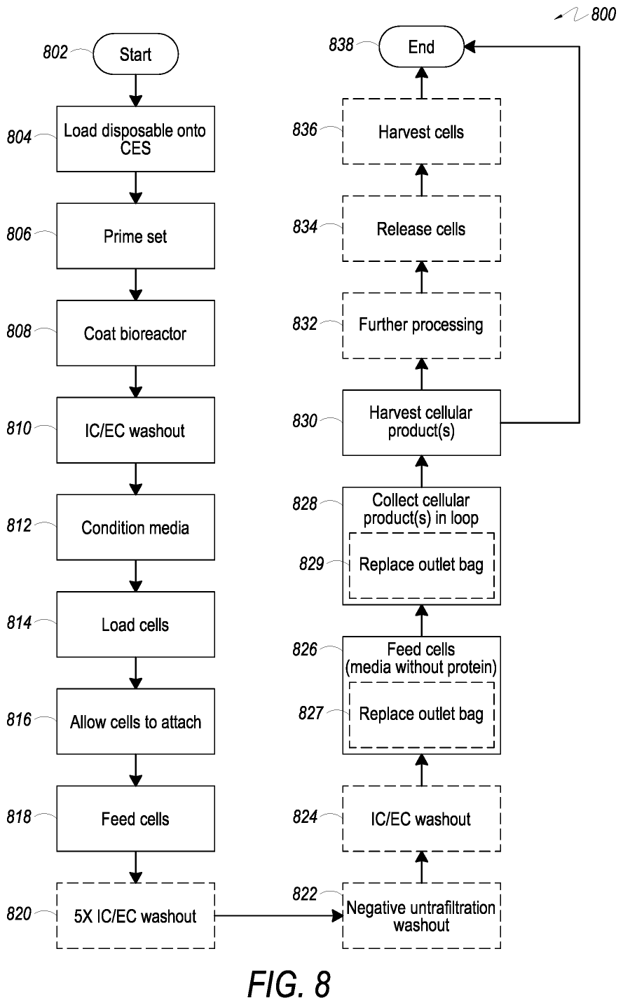

Next, FIG. 9 illustrates example operational steps 900 of a process for producing, isolating, and/or collecting released agents, e.g., EVs or viral vectors, etc., that may be used with a cell expansion system, such as CES 500 (FIG. 5) or CES 600 (FIG. 6), in accordance with embodiments of the present disclosure. START operation 902 is initiated, and process 900 proceeds to load the disposable tubing set 904 onto the cell expansion system. Next, the system may be primed 906. In an embodiment, a user or an operator, for example, may provide an instruction to the system to prime by selecting a task for priming, for example. In an embodiment, such task for priming may be a pre-programmed task. Process 900 then proceeds to coat the bioreactor 908, in which the bioreactor may be coated with a coating agent. For example, in embodiments, a reagent may be loaded into an IC loop until a reagent container is empty. The reagent may be chased from an air removal chamber into the IC loop, and the reagent may then be circulated in the IC loop. Any coating reagent known to those of skill in the art may be used, such as fibronectin or cryoprecipitate, for example. Once the bioreactor is coated, the IC/EC Washout task may be performed 910, in which fluid on the IC circulation loop and on the EC circulation loop may be replaced. The replacement volume may be determined by the number of IC Volumes and EC Volumes exchanged, according to an embodiment.

Next, to maintain the proper or desired gas concentration across the fibers in the bioreactor membrane, the condition media task 912 may be executed to allow the media to reach equilibrium with the provided gas supply before cells are loaded into the bioreactor. For example, rapid contact between the media and the gas supply provided by the gas transfer module or oxygenator is provided by using a high EC circulation rate. The system may then be maintained in a proper or desired state until a user or operator, for example, is ready to load cells into the bioreactor. In an embodiment, the system may be conditioned with complete media, for example. Complete media may be any media source used for cell growth. In an embodiment, complete media may comprise alpha-MEM (α-MEM) and fetal bovine serum (FBS), for example. Any type of media known to those of skill in the art may be used.

Process 900 next proceeds to loading cells 914 into the bioreactor from a cell inlet bag, for example. In an embodiment, the cells may be loaded into the bioreactor from the cell inlet bag until the bag is empty. Cells may then be chased from the air removal chamber to the bioreactor. In embodiments that utilize larger chase volumes, cells may be spread and move toward the IC outlet. The distribution of cells may be promoted across the membrane via IC circulation, such as through an IC circulation pump, with no IC inlet, for example. Further, the cells, e.g., adherent cells, may be allowed to attach 916 to the hollow fibers and be fed 918. In an embodiment, in allowing the cells to attach 916, adherent cells are enabled to attach to the bioreactor membrane while allowing flow on the EC circulation loop with the pump flow rate to the IC loop set to zero. The cells may grow/expand 920. In an embodiment, the cells may expand for three (3) to four (4) days. In another embodiment, the cells may expand for a period of time to achieve a particular desired number of cell doublings. In another embodiment, the cells may expand for a period of time to reach confluence. In an embodiment, the cells may be expanded for about seven (7) to about eight (8) days prior to collecting EVs, e.g., exosomes. Any time period may be used in accordance with embodiments of the present disclosure.

Next, process 900 proceeds to query 922, in which it is determined whether it is desired to collect cellular products, e.g., EVs or viral vectors, etc., released by the cells into the conditioned media during the growth/expansion of the cells 920. For example, it may be desired to begin isolating or purifying released agents for collection after a particular or defined number of cell doublings, such as two doublings, three doublings, four doublings, five doublings, six or more cell doublings, etc. For example, in an embodiment, it may be desired to begin isolating and/or collecting released agents after a minimum of two cell doublings. In another embodiment, it may be desired to begin purifying and/or collecting released agents after a minimum of five cell doublings, for example. In an embodiment, no defined number of cell doublings may be set before beginning an isolation and/or collection of released agents. Any number of cell doublings, defined time period, and/or other indicator may be used as understood by a person of skill in the art.

Returning to query 922, if it is desired to isolate and/or collect released agent(s), process 900 branches “yes” to optional wash out and replace step 924. In an embodiment, a wash out of serum-containing media may occur, in which such media may be replaced with base media, for example, 924. In an embodiment, such washout procedure may comprise one or more of the following step(s): (1) a 5× IC EC washout; (2) a negative ultrafiltration washout with phosphate buffered saline (PBS) to remove as much serum, if any, as possible from the bioreactor; and/or (3) a 2.5× IC EC washout with base media to replenish any metabolites lost during the PBS washouts. In an embodiment, all of the above-listed (1)-(3) steps are performed. In another embodiment, only one of the above-listed (1)-(3) steps is performed. In another embodiment, two of the above-listed (1)-(3) steps are performed. Further, any order of steps may be used in accordance with embodiments. In yet further embodiments, no washout occurs at step 924, and such step 924 may be optional where, for example, no animal-derived serum is used, only human-derived serum is used, serum-free media is used, and/or there are no other such additional protein source(s) to be removed, for example.

Following the washout of any serum-containing media 924 from the bioreactor, the IC outlet may be closed 926 by closing the IC outlet valve to allow the concentration of agents released by the cells to increase in the bioreactor. As a part of such step 926, the outlet or waste bag(s) may optionally be replaced 928. Such replacement of the outlet or waste bag(s) allows for monitoring or testing of the bag's contents to be performed to monitor glucose/lactate amounts, determine if any released agents are crossing the membrane (since the IC outlet is closed), etc. As noted above, released agents, e.g., EVs or viral vectors, etc., produced by the cells may be too large to diffuse through the membrane, e.g., their molecular weights are too large. Where the IC outlet is closed, the released agents, e.g., EVs, may be maintained on the IC side of the bioreactor during expansion (or defined collection period) where the released agent, e.g., EV or viral vector, etc., concentration is continuously increased, in accordance with embodiments. In an embodiment, step 928 occurs simultaneously with the closing of the IC outlet. In another embodiment, step 928 occurs after closing the IC outlet. In yet another embodiment, step 928 occurs prior to closing the IC outlet. In other embodiments, the outlet bag may not be replaced where no testing/monitoring or other particular use of the outlet bag is desired after the closing of the IC outlet. While the IC outlet is referred to as being closed in process 900, it should be noted that the EC outlet may be closed in other embodiments where cell growth may occur on the EC side, for example.

Next, process 900 proceeds to feed the cells 930 and collect released agents 932, in which media without protein may be added to the EC side of the bioreactor and may be diffused through the semi-permeable membrane to the IC side. In such embodiment, the EC inlet comprises EC media, e.g., media without protein, for providing nutrients to the cells while the released agent(s) are being allowed to concentrate. In such embodiment, there may be no IC inlet, for example. Instead, the semi-permeable hollow fibers of the bioreactor may allow essential nutrients (e.g., glucose) to reach the cells by continuous perfusion, and metabolic waste products (e.g., lactate) may be actively removed and exits the system via diffusion (EC outlet open). In another embodiment, the cells may be fed by adding media on the IC side. In an embodiment, base media may be used for about forty-eight (48) hours to supplement the cells. In another embodiment, base media may be used for about twenty-four (24) hours to about seventy-two (72) hours to supplement the cells. In embodiments, the collecting, or concentrating, of the released agent, e.g., EV or viral vector, etc., in the IC loop (IC outlet closed 926) may occur for a defined time period, e.g., about forty-eight (48) hours, or, in other embodiments, for about twenty-four (24) hours to about seventy-two (72) hours, for example. In other embodiments, such feeding with base media and collecting of the released particle(s) may occur for greater than about seventy-two (72) hours, such as, for example, about seventy-two (72) hours to about ninety-six (96) hours. In another embodiment, such feeding with a second media and collecting of the released particle(s) may occur for about seventy-two (72) hours to about one hundred and twenty (120) hours. In yet other embodiments, such feeding and collecting may occur for a time period of less than twenty-four (24) hours.

When it is determined to begin harvesting the collected or concentrated released agent(s) (such as after about forty-eight (48) hours, or another time period, for example), the valve(s) for harvest may be opened 934, and the released agent(s) may be harvested 936 from the IC side of the bioreactor to a harvest container(s), according to an embodiment. In an embodiment, the IC valve for the IC outlet may be opened to allow for harvesting. In another embodiment, the harvest valve may be opened to allow for harvesting to the harvest bag(s) or harvest container(s). In an embodiment, such harvesting may occur at the end of the entire process. In another embodiment, such harvesting may occur at defined interval(s). In an embodiment, released agent(s), e.g., EVs or viral vectors, etc., may be transferred in suspension from the intracapillary circulation loop in the bioreactor to a harvest bag or harvest container. In an embodiment, media without protein may be used in such harvest task.

Following the harvesting of the released agent(s) 936, process 900 may optionally proceed to allow for further processing 944. Such further processing 944 may include processing for assay(s), in an embodiment. In another embodiment, such further processing 944 may include further concentrating of the released agent(s) from the media collected in the harvest bag(s). In another embodiment, such further processing 944 may include separating the released agent(s) from other components in the bag, such as cells where suspension cells may be grown and harvested with the released agent(s). In an embodiment, such further processing 944 may include further isolating and/or characterization of the released agent(s). From optional further processing step 944, process 900 may terminate at END operation 946 where there is no desire to harvest cells, e.g., any adherent cells, remaining in the bioreactor. In another embodiment, process 900 may proceed directly from harvest agent(s) 936 to terminate at END operation 946 where no further processing 944 or harvesting of the cells 940 is desired.

Returning to step 944, if it is desired to harvest the cells, e.g., any adherent or attached cells, for example, remaining in the bioreactor, process 900 proceeds to optional release cells step 938. Alternatively, process 900 may proceed directly to optional release cells step 938 from harvest agent(s) 936 where no further processing 944 is desired, and it is desired to release cells 938 from the bioreactor. Attached cells may be released 938 from the membrane of the bioreactor and suspended in the IC loop, for example. In embodiments, an IC/EC washout task in preparation for adding a reagent to release the cells may be performed as part of operation 938. For example, IC/EC media may be replaced with a phosphate buffered saline (PBS) to remove protein, calcium (Ca²⁺), and magnesium (Mg²⁺) in preparation for adding trypsin, or another chemical-releasing agent, to release any adherent cells. A reagent may be loaded into the system until the reagent bag is empty. The reagent may be chased into the IC loop, and the reagent may be mixed within the IC loop. Following the release of any adherent cells, harvest operation 940 transfers the cells in suspension from the IC circulation loop, for example, including any cells remaining in the bioreactor, to a harvest bag(s) or container(s). Finally, the disposable set may be optionally unloaded 942 as a part of process 900 from the cell expansion system, and process 900 then terminates at END operation 946.

With respect to the processes illustrated in FIGS. 7, 8, and 9, the operational steps depicted are offered for purposes of illustration and may be rearranged, combined into other steps, used in parallel with other steps, etc., according to embodiments of the present disclosure. Fewer or additional steps may be used in embodiments without departing from the spirit and scope of the present disclosure. Also, steps (and any sub-steps), such as priming, coating a bioreactor, loading cells, for example, may be performed automatically in some embodiments, such as by a processor executing pre-programmed tasks stored in memory, in which such steps are provided merely for illustrative purposes.

Examples and further description of tasks and protocols, including custom tasks and pre-programmed tasks, for use with a cell expansion system are provided in U.S. patent application Ser. No. 13/269,323 (“Configurable Methods and Systems of Growing and Harvesting Cells in a Hollow Fiber Bioreactor System,” filed Oct. 7, 2011) and U.S. patent application Ser. No. 13/269,351 (“Customizable Methods and Systems of Growing and Harvesting Cells in a Hollow Fiber Bioreactor System,” filed Oct. 7, 2011), which applications are hereby incorporated by reference herein in their entireties for all that they teach and for all purposes.

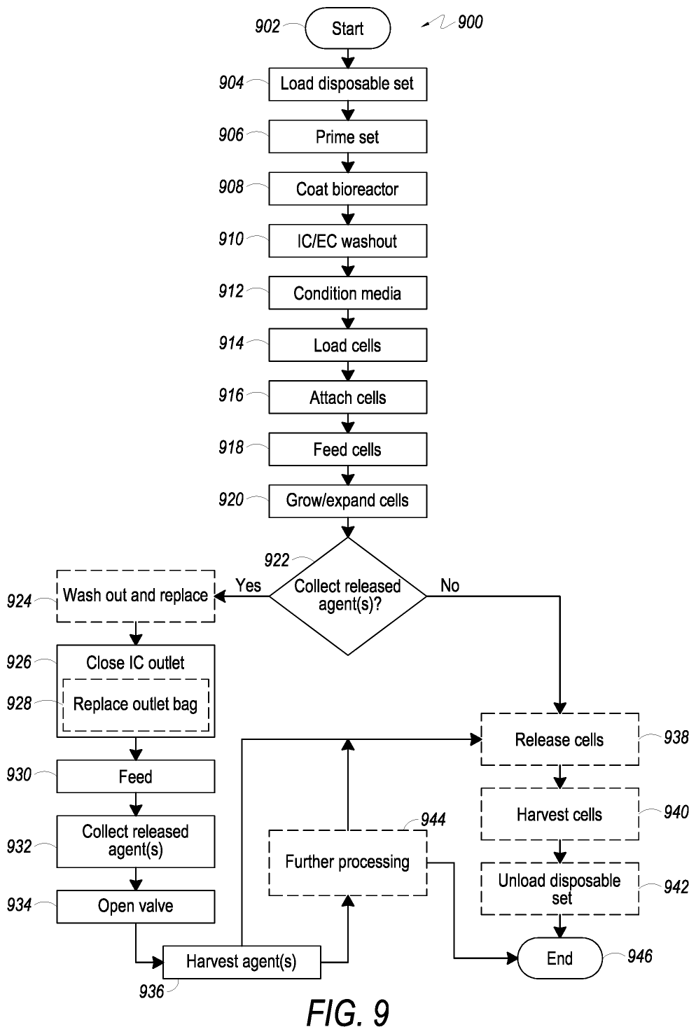

Next, FIG. 10 illustrates example components of a computing system 1000 upon which embodiments of the present disclosure may be implemented. Computing system 1000 may be used in embodiments, for example, where a cell expansion system uses a processor to execute tasks, such as custom tasks or pre-programmed tasks performed as part of processes such as processes 700, 800, and 900 described above. In embodiments, pre-programmed tasks may include, follow “IC/EC Washout” and/or “Feed Cells,” for example.

The computing system 1000 may include a user interface 1002, a processing system 1004, and/or storage 1006. The user interface 1002 may include output device(s) 1008, and/or input device(s) 1010 as understood by a person of skill in the art. Output device(s) 1008 may include one or more touch screens, in which the touch screen may comprise a display area for providing one or more application windows. The touch screen may also be an input device 1010 that may receive and/or capture physical touch events from a user or operator, for example. The touch screen may comprise a liquid crystal display (LCD) having a capacitance structure that allows the processing system 1004 to deduce the location(s) of touch event(s), as understood by those of skill in the art. The processing system 1004 may then map the location of touch events to UI elements rendered in predetermined locations of an application window. The touch screen may also receive touch events through one or more other electronic structures, according to embodiments. Other output devices 1008 may include a printer, speaker, etc. Other input devices 1010 may include a keyboard, other touch input devices, mouse, voice input device, etc., as understood by a person of skill in the art.

Processing system 1004 may include a processing unit 1012 and/or a memory 1014, according to embodiments of the present disclosure. The processing unit 1012 may be a general purpose processor operable to execute instructions stored in memory 1014. Processing unit 1012 may include a single processor or multiple processors, according to embodiments. Further, in embodiments, each processor may be a multi-core processor having one or more cores to read and execute separate instructions. The processors may include general purpose processors, application specific integrated circuits (ASICs), field programmable gate arrays (FPGAs), other integrated circuits, etc., as understood by a person of skill in the art.

The memory 1014 may include any short-term or long-term storage for data and/or processor executable instructions, according to embodiments. The memory 1014 may include, for example, Random Access Memory (RAM), Read-Only Memory (ROM), or Electrically Erasable Programmable Read-Only Memory (EEPROM), as understood by a person of skill in the art. Other storage media may include, for example, CD-ROM, tape, digital versatile disks (DVD) or other optical storage, tape, magnetic disk storage, magnetic tape, other magnetic storage devices, etc., as understood by a person of skill in the art.

Storage 1006 may be any long-term data storage device or component. Storage 1006 may include one or more of the systems described in conjunction with the memory 1014, according to embodiments. The storage 1006 may be permanent or removable. In embodiments, storage 1006 stores data generated or provided by the processing system 1004.

### EXAMPLES

Some examples of methods/processes/protocols/configurations that may implement aspects of the embodiments are described below. Although specific features may be described in these examples, they are provided merely for illustrative and descriptive purposes. The present invention is not limited to the examples provided below.

### Example 1

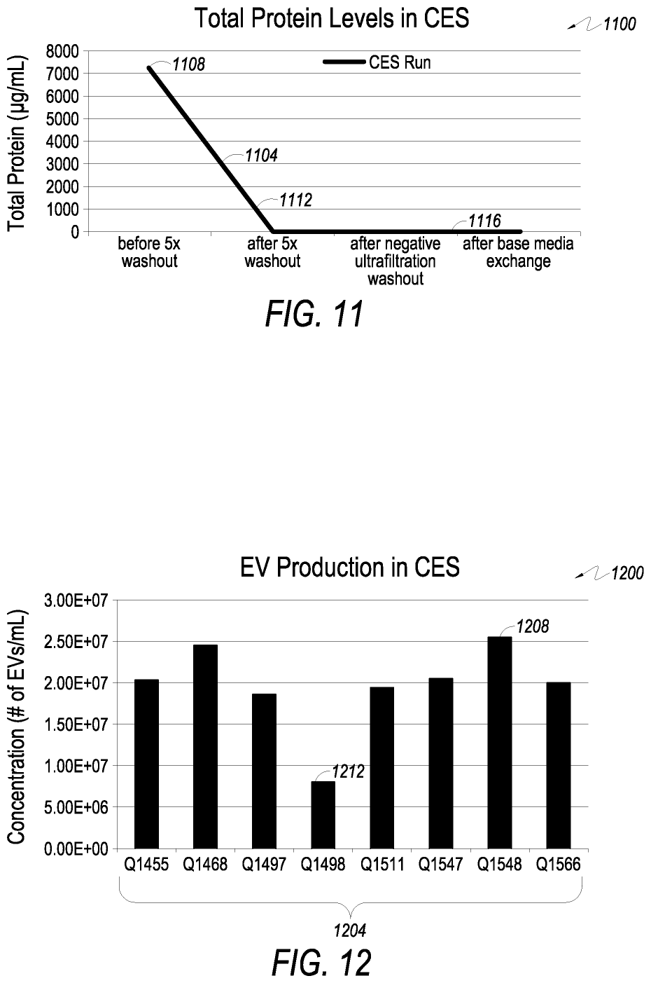

Example results of expanding cells and collecting extracellular particles, e.g., EVs, through implementation of the above systems and methods are shown in graph 1200 of FIG. 12. The graph 1200 shows a series 1204 of EV production runs using, for example, the methods 700, 800, and/or 900 and CES 500, 600 described above, according to embodiments. For example, such system production/collection may use the Quantum® Cell Expansion System (Quantum® System or Quantum® CES) manufactured by Terumo BCT, Inc. in Lakewood, Colo. As shown, various concentrations of EVs may be produced and collected using the processes and systems described above. The production runs may yield various concentrations of EVs, from a high value 1208 of over 2.5E+07 EVs/mL, to a low value 1212 of between 5.00E+06 EVs/mL and 1.00E+07 EVs/mL.

### Example 2

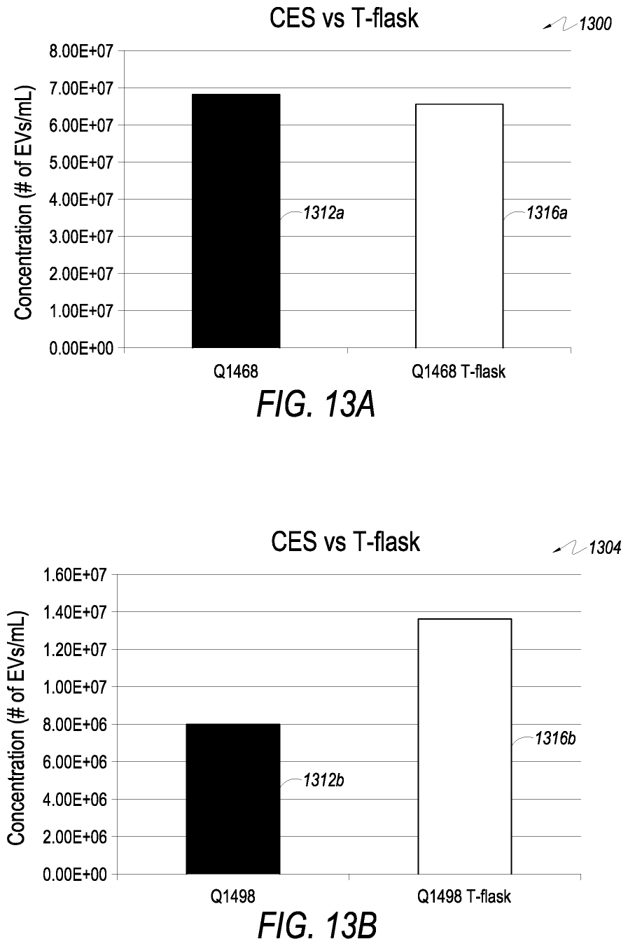

Example results of generating and/or collecting extracellular particles, e.g., EVs, with the above methods 700, 800, and/or 900 and/or with systems 500, 600 are shown in graphs 1300, 1304, and/or 1308, in FIGS. 13A, 13B, and/or 13C, according to embodiments. Each of the concentrations of EVs 1312a and/or 1312c that may be generated by the system 500, 600 may be near or more than the concentrations of EVs 1316a and/or 1316c generated in a 225 cm² tissue culture flask (T225), in which such flask may be seeded at a substantially similar cell density as the system 500, 600 and treated similarly for comparison, e.g., using similar cell densities and feedings. As shown in FIG. 13B, the concentration of EVs 1316b generated in a 225 cm² tissue culture flask (T225) may be more than the concentration of EVs 1312b generated by the system 500, 600, in which such flask may be seeded at a substantially similar seeding density as the system 500, 600 and treated similarly for comparison.

As an example, FIG. 13A shows possible results for generating and/or collecting EVs in a run numbered as “Q1468” 1312a, of a Quantum® Cell Expansion System, as compared to those EVs which may be produced and/or collected in a T-flask (“Q1468 T-flask”) 1316a seeded at a substantially similar seeding density and treated similarly for comparison. For example, a bioreactor in the Quantum® System (surface area of 21,000 cm²) for Q1468 may be loaded with 1.25E+08 cultured cells, while the Q1468 T-flask (surface area of 225 cm²) may be loaded with 9.24E+05 cultured cells, which may equate to loading both the Quantum® System and the T-flask with a substantially similar cell density. While the example results in FIG. 13A show a concentration between 6.00E+07 EVs/mL and 7.00E+07 EVs/mL for both 1312a (Quantum® System) and 1316a (T-flask), graph 1300 shows a higher concentration of EVs 1312a collected from the Quantum® System as compared to the concentration of EVs 1316a collected from the T-flask (Q1468 T-flask).

The example results in FIGS. 13A and 13C, for example, may show higher concentrations of EVs obtained by the system production/collection (for example, using the Quantum® Cell Expansion System manufactured by Terumo BCT, Inc. in Lakewood, Colo.) than the concentrations obtained by using similar cell loads (for example, similar cell densities) and feeding amounts in a conventional T-flask. Using CES 500, 600 may be less labor-intensive due to the possible automation of several functions and may provide higher numbers of EVs in some embodiments due to a greater surface area to grow cells.

### Example 3

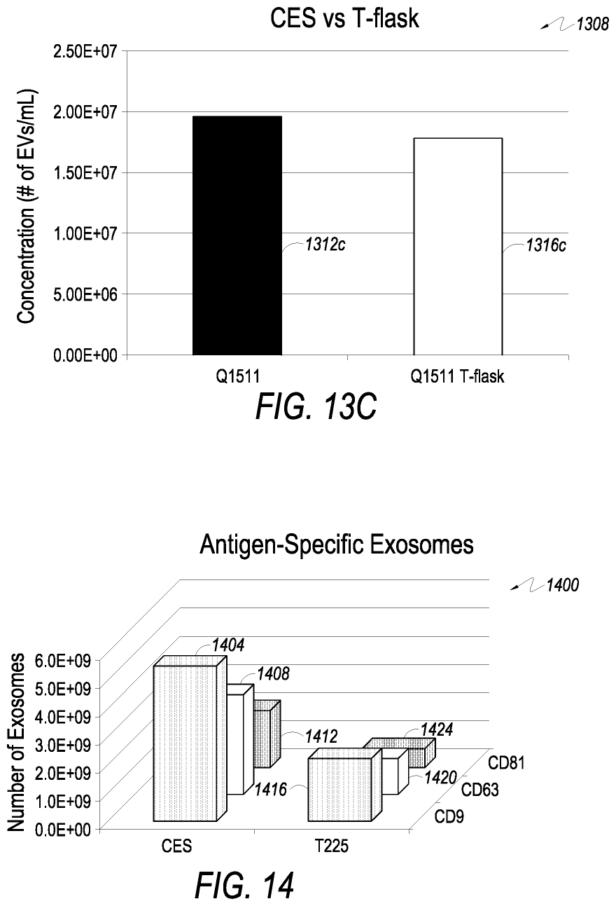

Example results of generating and/or collecting antigen-specific extracellular particles, e.g., EVs, with, for example, the above methods 700, 800, and/or 900 and/or with systems 500, 600, are shown in FIG. 14, according to embodiments. As shown in FIG. 14, example results may be obtained from a representative sample of purified exosomes obtained from a conventional T-flask and from a system 500, 600, such as the Quantum® Cell Expansion System manufactured by Terumo BCT, Inc. in Lakewood, Colo. As shown in graph 1400, the types of antigen-specific exosomes can include CD9, CD63, and/or CD81. A number of CD9 exosomes produced and collected from a system 500, 600 may be shown in column 1404; a number of CD63 exosomes produced and collected from a system 500, 600 may be shown in column 1408; and a number of CD81 exosomes produced and collected from a system 500, 600 may be shown in column 1412. Further, cells may be seeded in a 225 cm² tissue culture flask (T225) at a substantially similar cell density as the system 500, 600, e.g., Quantum® System, and treated similarly for comparison. As shown in graph 1400, a number of CD9 exosomes produced and collected from a tissue culture flask (T225) may be shown in column 1416; a number of CD63 exosomes produced and collected from a tissue culture flask (T225) may be shown in column 1420; and a number of CD81 exosomes produced and collected from a tissue culture flask (T225) may be shown in column 1424. As shown in FIG. 14, the number of exosomes produced and collected by the system 500, 600, with respect to each antigen may be higher than the number of exosomes produced and collected by the tissue culture flask (T225) with respect to each antigen. For example, FIG. 14 may show that the quantities of each of the antigen-specific exosomes from the CES harvest may be observed to be 2-3 times higher than the T225 harvest, according to an embodiment.

### Example 4

As explained with respect to FIG. 8, a Quantum® System, e.g., CES 500 and/or 600, may be primed with PBS and coated overnight with 5 mg of FN. The following day, the system may undergo a 2.5× IC EC washout and may be conditioned with complete media (αMEM with GlutaMAX plus 10% FBS). Pre-selected MSC may be seeded into the bioreactor and expanded for 4-5 days. At this point, the system may undergo a 5× IC EC washout and a negative ultrafiltration washout with PBS to remove as much serum as possible from the bioreactor. A 2.5× IC EC washout with base media (αMEM with GlutaMAX only) may then be performed to replenish metabolites lost during the PBS washouts. Base media may be used to supplement the cells for forty-eight (48) hours while the conditioned media may be collected into a harvest bag. A flask may also be seeded at a substantially similar seeding density as the bioreactor and treated similarly to the Quantum® System for comparison purposes.

Possible results from, for example, the above methods 700, 800, and/or 900 and/or systems 500, 600 used as part of EXAMPLE 4 may be observed to generate 4.71×10¹¹ total exosomes as a result of loading 9.9 million cells into the bioreactor 501, 601, expanding for six (6) days, the serum in the system being washed out, and the exosomes being collected in the IC loop for two (2) days. After the exosomes are collected, the exosomes may be harvested from the IC loop, in which 135 million MSCs may be observed to be recovered from the bioreactor 501, 601.

The embodiments of the disclosure may have one or more aspects, including, for example:

Embodiments and/or aspects of the invention can include a method of collecting a cellular product, the method comprising: loading cells into a bioreactor; feeding the cells with media; expanding the cells, wherein the cells release a cellular product; concentrating the released cellular product; and harvesting the concentrated cellular product from the bioreactor.

Any of the one or more above embodiments and/or aspects, wherein the released cellular product comprises an extracellular particle.

Any of the one or more above embodiments and/or aspects, wherein the extracellular particle comprises an extracellular vesicle.

Any of the one or more above embodiments and/or aspects, wherein the extracellular vesicle comprises an exosome.

Any of the one or more above embodiments and/or aspects, wherein the extracellular vesicle comprises a microvesicle.

Any of the one or more above embodiments and/or aspects, wherein the extracellular particle comprises a viral vector.

Any of the one or more above embodiments and/or aspects, wherein the harvested cellular product is collected into a bag.

Any of the one or more above embodiments and/or aspects, further comprising: replacing the media.

Any of the one or more above embodiments and/or aspects, wherein the media comprises serum.

Embodiments and/or aspects of the invention can include a cell expansion system comprising: a bioreactor, wherein the bioreactor comprises a hollow fiber membrane; a first fluid flow path having at least opposing ends, wherein the first fluid flow path is fluidly associated with an intracapillary portion of the hollow fiber membrane; a processor; a memory, in communication with and readable by the processor, and containing a series of instructions that, when executed by the processor, cause the processor to: receive a selection to feed cells; receive a selection to close an outlet of the intracapillary portion, wherein the closing an outlet of the intracapillary portion concentrates particles released from the cells in the intracapillary portion; and conduct an operation to move the particles to a harvest bag.

Any of the one or more above embodiments and/or aspects, further comprising one or more of: conduct an operation to perform a 5× washout; conduct an operation to perform a negative ultrafiltration; and/or conduct an operation to perform a washout.

Any of the one or more above embodiments and/or aspects, wherein the particles released from the cells comprise extracellular particles.

Any of the one or more above embodiments and/or aspects, wherein the extracellular particles comprise exosomes.

Any of the one or more above embodiments and/or aspects, wherein the extracellular particles comprise microvesicles.

Any of the one or more above embodiments and/or aspects, further comprising: receive a selection to replace fluid in the intracapillary portion and in an extracapillary portion of the hollow fiber membrane.

Any of the one or more above embodiments and/or aspects, wherein the replacing of the fluid extracts protein from a first media used to feed the cells.

Any of the one or more above embodiments and/or aspects, wherein a second media without protein replaces the first media to feed the cells.

Any of the one or more above embodiments and/or aspects, further comprising conduct an operation to perform a test of the first and/or second media in the bioreactor to determine if the protein has been removed.

Embodiments and/or aspects of the invention can include a cell expansion system comprising: a bioreactor, wherein the bioreactor comprises a hollow fiber membrane; a first fluid flow path having a first inlet and a first outlet at at least opposing ends of the bioreactor, wherein the first fluid flow path is fluidly associated with an intracapillary portion of the hollow fiber membrane; a second fluid flow path having a second inlet and a second outlet, wherein the second fluid flow path is fluidly associated with an extracapillary portion of the hollow fiber membrane; a first connection port fluidly associated with the first fluid flow path, wherein a first bag attached to the first connection port introduces cells to the bioreactor; a second connection port fluidly associated with the first fluid flow path, wherein a second bag containing a first media containing protein is connected to the second connection port to provide the first media to the bioreactor through the first fluid flow path to feed the cells until a predetermined number of cell doublings has occurred; a third connection port fluidly associated with the first fluid flow path and the second fluid path, wherein a third bag containing a second media is connected to the third connection port to provide the second media to the bioreactor through the first fluid flow path and the second fluid path to wash out the first media from the bioreactor; a fourth connection port fluidly associated with the second fluid flow path, wherein, after the washout, a fourth bag containing a third media without protein is connected to the fourth connection port to feed the cells, wherein the first outlet of the first fluid flow path is closed when feeding the cells with the third media to concentrate particles released from the cells in the intracapillary portion of the bioreactor; and a harvest bag fluidly associated with the first fluid flow path, wherein the concentrated particles are moved into the harvest bag.

Embodiments and/or aspects of the invention can include a method for generating cellular particles in a cell expansion system, the method comprising: priming the cell expansion system, wherein the cell expansion system comprises: a bioreactor, wherein the bioreactor comprises: a hollow fiber membrane having an intracapillary portion and an extracapillary portion; a first fluid flow path having a first inlet and a first outlet at at least opposing ends of the bioreactor, wherein the first fluid flow path is fluidly associated with an intracapillary portion of the hollow fiber membrane; a second fluid flow path having a second inlet and a second outlet, wherein the second fluid flow path is fluidly associated with an extracapillary portion of the hollow fiber membrane; a first connection port fluidly associated with the first fluid flow path; a second connection port fluidly associated with the first fluid flow path; a third connection port fluidly associated with the first fluid flow path and the second fluid path; a fourth connection port fluidly associated with the second fluid flow path; and a harvest bag; connecting a first bag to the first connection port to introduce cells to the bioreactor; connecting a second bag containing a first media containing protein to the second connection port to provide the first media to the bioreactor through the first fluid flow path to feed the cells until a predetermined number of cell doublings has occurred; after the predetermined number of cell doublings has occurred, connecting a third bag containing a second media to the third connection port to provide the second media to the bioreactor through the first fluid flow path and the second fluid path to wash out the first media from the bioreactor; after the washout, closing the first outlet of the first fluid flow path to concentrate particles released from the cells in the intracapillary portion of the bioreactor; connecting a fourth bag containing a third media without protein to the fourth connection port to feed the cells; connecting the harvest bag to the first fluid flow path to harvest; and harvesting the concentrated particles into the harvest bag.

Embodiments and/or aspects of the invention can include any of the one or more above embodiments and/or aspects in combination.

Embodiments and/or aspects of the invention can include a means for performing any of the one or more above embodiments and/or aspects.

While example embodiments and applications of the present invention have been illustrated and described, it is to be understood that the invention is not limited to the precise configuration and resources described above. Various modifications, changes, and variations apparent to those skilled in the art may be made in the arrangement, operation, and details of the methods and systems of the present invention disclosed herein without departing from the scope of the present invention.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

It will be apparent to those skilled in the art that various modifications and variations may be made to the methods and structure of the present invention without departing from its scope. Thus, it should be understood that the invention is not to be limited to the specific examples given. Rather, the invention is intended to cover modifications and variations within the scope of the following claims and their equivalents.

What is claimed is:

1. A method of collecting a cellular product, the method comprising:
loading cells into a bioreactor;
feeding the cells with media;
expanding the cells, wherein the cells release a cellular product;
concentrating the released cellular product; and
harvesting the concentrated cellular product from the bioreactor.

2. The method of claim 1, wherein the released cellular product comprises an extracellular particle.

3. The method of claim 2, wherein the extracellular particle comprises an extracellular vesicle.

4. The method of claim 3, wherein the extracellular vesicle comprises an exosome.

5. The method of claim 3, wherein the extracellular vesicle comprises a microvesicle.

6. The method of claim 2, wherein the extracellular particle comprises a viral vector.

7. The method of claim 1, wherein the harvested cellular product is collected into a bag.

8. The method of claim 1, further comprising:
replacing the media.

9. The method of claim 1, wherein the media comprises serum.

10. A cell expansion system comprising:
a bioreactor, wherein the bioreactor comprises a hollow fiber membrane;
a first fluid flow path having at least opposing ends, wherein the first fluid flow path is fluidly associated with an intracapillary portion of the hollow fiber membrane;
a processor;
a memory, in communication with and readable by the processor, and containing a series of instructions that, when executed by the processor, cause the processor to:
receive a selection to feed cells;
receive a selection to close an outlet of the intracapillary portion, wherein the closing an outlet of the intracapillary portion concentrates particles released from the cells in the intracapillary portion; and
conduct an operation to move the particles to a harvest bag.

11. The cell expansion system of claim 10, further comprising one or more of:
conduct an operation to perform a 5× washout;
conduct an operation to perform a negative ultrafiltration; and/or
conduct an operation to perform a washout.

12. The cell expansion system of claim 10, wherein the particles released from the cells comprise extracellular particles.

13. The cell expansion system of claim 12, wherein the extracellular particles comprise exosomes.

14. The cell expansion system of claim 12, wherein the extracellular particles comprise microvesicles.

15. The cell expansion system of claim 10, further comprising:
receive a selection to replace fluid in the intracapillary portion and in an extracapillary portion of the hollow fiber membrane.

16. The cell expansion system of claim 15, wherein the replacing of the fluid extracts protein from a first media used to feed the cells.

17. The cell expansion system of claim 16, wherein a second media without protein replaces the first media to feed the cells.

18. The cell expansion system of claim 17, further comprising:
conduct an operation to perform a test of the first and/or second media in the bioreactor to determine if the protein has been removed.

19. A cell expansion system comprising:
a bioreactor, wherein the bioreactor comprises a hollow fiber membrane;
a first fluid flow path having a first inlet and a first outlet at at least opposing ends of the bioreactor, wherein the first fluid flow path is fluidly associated with an intracapillary portion of the hollow fiber membrane;
a second fluid flow path having a second inlet and a second outlet, wherein the second fluid flow path is fluidly associated with an extracapillary portion of the hollow fiber membrane;
a first connection port fluidly associated with the first fluid flow path, wherein a first bag attached to the first connection port introduces cells to the bioreactor;
a second connection port fluidly associated with the first fluid flow path, wherein a second bag containing a first media containing protein is connected to the second connection port to provide the first media to the bioreactor through the first fluid flow path to feed the cells until a predetermined number of cell doublings has occurred;
a third connection port fluidly associated with the first fluid flow path and the second fluid path, wherein a third bag containing a second media is connected to the third connection port to provide the second media to the bioreactor through the first fluid flow path and the second fluid path to wash out the first media from the bioreactor;
a fourth connection port fluidly associated with the second fluid flow path, wherein, after the washout, a fourth bag containing a third media without protein is connected to the fourth connection port to feed the cells, wherein the first outlet of the first fluid flow path is closed when feeding the cells with the third media to concentrate particles released from the cells in the intracapillary portion of the bioreactor; and
a harvest bag fluidly associated with the first fluid flow path, wherein the concentrated particles are moved into the harvest bag.

20. A method for generating cellular particles in a cell expansion system, the method comprising:
priming the cell expansion system, wherein the cell expansion system comprises:
a bioreactor, wherein the bioreactor comprises:
a hollow fiber membrane having an intracapillary portion and an extracapillary portion;
a first fluid flow path having a first inlet and a first outlet at at least opposing ends of the bioreactor, wherein the first fluid flow path is fluidly associated with an intracapillary portion of the hollow fiber membrane;
a second fluid flow path having a second inlet and a second outlet, wherein the second fluid flow path is fluidly associated with an extracapillary portion of the hollow fiber membrane;
a first connection port fluidly associated with the first fluid flow path;
a second connection port fluidly associated with the first fluid flow path;
a third connection port fluidly associated with the first fluid flow path and the second fluid path;
a fourth connection port fluidly associated with the second fluid flow path; and
a harvest bag;
connecting a first bag to the first connection port to introduce cells to the bioreactor;
connecting a second bag containing a first media containing protein to the second connection port to provide the first media to the bioreactor through the first fluid flow path to feed the cells until a predetermined number of cell doublings has occurred;
after the predetermined number of cell doublings has occurred, connecting a third bag containing a second media to the third connection port to provide the second media to the bioreactor through the first fluid flow path and the second fluid path to wash out the first media from the bioreactor;
after the washout, closing the first outlet of the first fluid flow path to concentrate particles released from the cells in the intracapillary portion of the bioreactor;
connecting a fourth bag containing a third media without protein to the fourth connection port to feed the cells;
connecting the harvest bag to the first fluid flow path to harvest; and
harvesting the concentrated particles into the harvest bag.
